(19) United States
(12) Patent Application Publication
(10) Pub. No.: US 2005/0059085 A1
(43) Pub. Date: Mar. 17, 2005

(54) Crystal of bacterial core RNA polymerase with rifampicin and methods of use thereof

(75) Inventors: Seth A. Darst, New York, NY (US); Elizabeth Campbell, New York, NY (US)

Correspondence Address: FOLEY HOAG, LLP;PATENT GROUP, WORLD TRADE CENTER WEST, 155 SEAPORT BLVD, BOSTON, MA, 02110, US

(73) Assignee: The Rockefeller University, New York, NY (US)

(21) Appl. No.: 10/783,206

(22) Filed: Feb. 20, 2004

Related U.S. Application Data
(62) Division of application No. 09/802,755, filed on Mar. 9, 2001, now abandoned.

(51) Int. Cl. G01N 33/53; G06F 19/00; G01N 33/48; G01N 33/50; C07D 309/30

(52) U.S. Cl. 435/7.1; 702/19

(57) ABSTRACT

A detailed three-dimensional structure of rifampicin bound to a core bacterial RNA polymerase (Rif-RNAP) is provided. Crystals of the Rif-RNAP are also included in the invention. The present invention further provides procedures for identifying agents that can inhibit bacterial proliferation through the use of rational drug design predicated on the crystals and crystallographic data disclosed.

### GOVERNMENTAL SUPPORT

The research leading to the present invention was supported, at least in part, by grants from NIH, Grants GM 53759, GM 20470, GM 61898, GM 49242 and GM 30717. Accordingly, the Government may have certain rights in the invention.

### REFERENCE TO TABLE SUBMITTED ON COMPACT DISC

Two compact discs are included with the instant filing which contain identical material. The material on the compact disc is hereby incorporated by reference in its entirety under 37 CFR § 1.77(b)(4). The compact discs contain a single file, dated Mar. 9, 2001, labeled RNAP_RIF_final.pdb which is an ASCII text file that is 1.46 MB (1,536,303 bytes), 1,540,096 bytes used. The compact discs contain the structural coordinates for the Rif-RNAP complex with the Thermus aquaticus core RNA polymerase which is also included in a hard copy as Table 2 in the Appendix, following the Sequence Listing.

### FIELD OF THE INVENTION

The present invention provides a crystal of a binding complex between rifampicin and a bacterial core RNA polymerase from Thermus aquaticus. The three-dimensional structural information is included in the invention. The present invention provides procedures for identifying agents that can inhibit bacterial cell growth through the use of rational drug design predicated on the crystallographic data.

### BACKGROUND OF THE INVENTION

RNA in all cellular organisms is synthesized by a complex molecular machine, the DNA-dependent RNA polymerase (RNAP). In its simplest bacterial form, the enzyme comprises at least 4 subunits with a total molecular mass of around 400 kDa.

The eukaryotic enzymes comprise upwards of a dozen subunits with a total molecular mass of around 500 kDa. The essential core component of the RNAP (subunit composition α₂ββ′ω) is evolutionarily conserved from bacteria to man [Archambault and Friesen, Microbiological Reviews, 57:703-724 (1993)]. Sequence homologies point to structural and functional homologies, making the simpler bacterial RNAPs excellent model systems for understanding the multisubunit cellular RNAPs in general.

The basic elements of the transcription cycle were elucidated through study of the prokaryotic system. In this cycle, the RNAP, along with other factors, locates specific sequences called promoters within the double-stranded DNA, forms the open complex by melting a portion of the DNA surrounding the transcription start site, initiates the synthesis of an RNA chain, and elongates the RNA chain completely processively while translocating itself and the melted transcription bubble along the DNA template. Finally it releases itself and the completed transcript from the DNA when a specific termination signal is encountered. The current view is that the transcribing RNAP contains sites for binding the DNA template as well as forming and maintaining the transcription bubble, binding the RNA transcript, and binding the incoming nucleotide-triphosphate substrate.

From the initial indications of DNA-dependent RNAP activity from a number of systems, [Weiss and Gladstone, J. Am. Chem. Soc., 81:4118-4119 (1959)]; Hurwitz et al., Biochem. Biophys. Res. Commun., 3:15 (1960); Stevens, Biochem. Biophys. Res. Commun., 3:92 (1960); Huang et al., Biochem. Biophys. Res. Commun., 3:689 (1960); and Weiss and Nakamoto, J. Biol. Chem., 236:PC 19 (1961)], and the isolation of the RNAP enzyme from bacterial sources [Chamberlin and Berg, Proc. Natl. Acad. Sci. USA, 48:81-94 (1962)], a wealth of biochemical, biophysical, and genetic information has accumulated on RNAP and its complexes with nucleic acids and accessory factors. Nevertheless, the enzyme itself, in terms of its structure/function relationship, remains a black box. An essential step towards understanding the mechanism of transcription and its regulation is to determine three-dimensional structures of RNAP and its complexes with DNA, RNA, and regulatory factors [von Hippel et al., Annual reviews of Biochemistry, 53:389-446 (1984); Erie et al., Annual Review of Biophysics & Biomolecular Structure, 21:379-415 (1992); Sentenac et al., Transcriptional Regulation, in Cold Spring Harbor Laboratory 27-54, Cold Spring Harbor, eds. McKnight and Yamamoto (1992); Gross et al., Philosophical Transactions of the Royal Society of London—Series B:Biological Sciences, 351:475-482 (1996); and Nudler, J. Mol. Biol., 288:1-12 (1999)].

The key feature of low-resolution structures of bacterial and eukaryotic RNAPs, provided by electron crystallography, is a thumb-like projection surrounding a groove or channel that is an appropriate size for accommodating double-helical DNA [Darst et al., Nature, 340:730-732 (1989); Darst et al., Cell, 66:121-128 (1991); Schultz et al., EMBO J., 12:2601-2607 (1993); Polyakov et al., Cell, 83:365-373 (1995); Darst et al., J. Structural Biol., 124:115-122 (1998); and Darst et al., Cold Spring Harbor Symp. Quant. Biol., 63:269-276 (1998)].

Bacterial infections remain among the most common and deadly causes of human disease. Infectious diseases are the third leading cause of death in the United States and the leading cause of death worldwide [Binder et al., Science 284:1311-1313 (1999)]. More particularly, each year there are 8-10 million new cases of tuberculosis (TB). TB is the leading cause of death in adults by an infectious agent [Raviglioni et al., JAMA 273:220-226 (1995); Shinnick, Current Topics in Microbiol. Immunol., Springer-Verlag Berlin Heidelberg, N.Y.(1996)] and is in near epidemic proportions in some parts of the world. Indeed, the World Health Organization declared TB to be a global public health emergency due to the rapid increase in multi-drug resistant strains of Mycobaterium tuberculosis [Raviglioni et al., JAMA 273:220-226 (1995)].

Rifampicin (Rif) [Sensi, Antibiot. Ann 1959-1960, 262-270 (1960); Sensi et al., Rev. Infect. Dis., 5 Supp.3:402406 (1983)] is one of the most potent and broad-spectrum antibiotics against bacterial pathogens and is a key component of anti-TB therapy. The introduction of rifampicin in 1968 greatly shortened the duration of chemotherapy necessary for successful treatment. Rifampicin diffuses freely into tissues, living cells, and bacteria, making it extremely effective against intracellular pathogens like M. tuberculosis [Shinnick, Current Topics in Microbiol. Immunol., Springer-Verlag Berlin Heidelberg, N.Y.(1996)]. However, bacteria develop resistance to rifampicin with high frequency, which has led the medical community in the United States to commit to a voluntary restriction of its use for treatment of TB or emergencies.

The bactericidal activity of rifampicin stems from its high-affinity binding to, and inhibition of, the bacterial DNA-dependent RNA polymerase [Hartmann et al., Biochim. Biophys. Acta 145:843-844 (1967)]. Mutations conferring rifampicin resistance (Rif^{R}) map almost exclusively to the rpoB gene (encoding the RNAP β subunit) in every organism tested, including E. coli [Ezekiel and Hutchins, Nature London 220:276-277(1968); Heil and Zillig, FEBS Lett. 11: 165-168 (1970); Wehrli et al., Biochem. Biophysic. Res. Comm., 32:284-288 (1968) and M. tuberculosis [Heep et al., Antimicrob. Agents Chemotherap. 44:1075-1077 (2000); Ramaswamy and Musser, Tubercle and Lung Disease 79:3-29 (1998)]. Comprehensive genetic analyses have provided molecular details of amino acid alterations in β subunit conferring Rif^{R} (see FIG. 1) [Jin and Gross, J. Molec. Biol, 202:45-58 1988; Lisitsyn et al., Bioorg Khim 10: 127-128 (1984); Lisitsyn et al., Molec. Gen. Genet., 196:173-174 (1984); Ovchinnikov et al., Molec. Gen. Genet. 190:344-348 (1983); Severinov et al., J. Biol. Chem., 268:14820-14825 (1993); Severinov et al., Molec. Gen. Genet., 244:120-126 (1994)].

Although, there was initial optimism in the middle of this century that diseases caused by bacteria would be quickly eradicated, it has become evident that the so-called “miracle drugs” are not sufficient to accomplish this task. Indeed, antibiotic resistant pathogenic strains of bacteria have become common-place, and bacterial resistance to the new variations of these drugs appears to be outpacing the ability of scientists to develop effective chemical analogs of the existing drugs [See, Stuart B. Levy, The Challenge of Antibiotic Resistance, in Scientific American, 46-53 (March, 1998)]. Therefore, new approaches to drug development are necessary to combat the ever-increasing number of antibiotic-resistant pathogens.

Classical penicillin-type antibiotics effect a single class of proteins known as autolysins. Thus, the development of new drugs which effect an alternative bacterial target protein would be desirable. Such a target protein ideally would be indispensable for bacterial survival. A enzyme such as bacterial RNAP would thus be a prime candidate for such drug development.

Therefore, there is a need to develop methods for identifying drugs that interfere with bacterial RNAP. Unfortunately, such identification has heretofore relied on serendipity and/or systematic screening of large numbers of natural and synthetic compounds. One superior method for drug screening relies on structure based rational drug design. In such cases, a three dimensional structure of the protein or peptide is determined and potential agonists and/or antagonists are designed with the aid of computer modeling [Bugg et al., Scientific American, Dec.: 92-98 (1993); West et al., TIPS, 16:67-74 (1995); Dunbrack et al., Folding & Design, 2:27-42 (1997)].

Therefore, there is a need for obtaining a crystal of the bacterial RNAP bound to an inhibitor that is amenable to high resolution X-ray crystallographic analysis. In addition, there is a need for determining the three-dimensional structure of the RNAP bound to that inhibitor. Furthermore, there, is a need for developing procedures of structure based rational drug design using such three-dimensional information. Finally, there is a need to employ such procedures to develop new anti-bacterial drugs.

The citation of any reference herein should not be construed as an admission that such reference is available as “Prior Art” to the instant application.

### SUMMARY OF THE INVENTION

The present invention provides crystals of RNA polymerase bound to an inhibitor. More particularly, the present invention provides crystals of the bacterial core RNA polymerase bound to rifampicin (the Rif-RNAP complex). In addition, the present invention also provides detailed three-dimensional structural data for the Rif-RNAP complex. The structural data obtained for the Rif-RNAP complex can be used for the rational design of drugs that inhibit bacterial cell proliferation. The present invention further provides methods of identifying and/or improving inhibitors of the bacterial core RNA polymerase which can be used in place of and/or in conjunction with other bacterial inhibitors including antibiotics.

One aspect of the present invention provides crystals of the bacterial core RNA polymerase bound to rifampicin that can effectively diffract X-rays for the determination of the atomic coordinates of the Rif-RNAP complex to a resolution of better than 5.0 Angstroms. In a preferred embodiment the crystal effectively diffracts X-rays for the determination of the atomic coordinates of the Rif-RNAP complex to a resolution of 3.5 Angstroms or better. In a particular embodiment the crystal of the Rif-RNAP complex effectively diffracts X-rays for the determination of the atomic coordinates to a resolution of 3.3 Angstroms or better.

In a particular embodiment the bacterial core RNA polymerase of the crystal is a thermophilic bacterial core RNA polymerase. In a preferred embodiment of this type the thermophilic bacterial core RNA polymerase is a Thermus aquaticus bacterial core RNA polymerase. Such a core RNA polymerase comprises β′ subunit, a β subunit, and a pair of α subunits. Preferably, the core RNA polymerase further comprises an co subunit. In a particular embodiment the β′ subunit has the amino acid sequence of SEQ ID NO: 1. In another embodiment the β subunit has the amino acid sequence of SEQ ID NO:2. In still another embodiment an α subunit has the amino acid sequence of SEQ ID NO:3. In still another embodiment an ω subunit has the amino acid sequence of SEQ ID NO:4.

In a preferred embodiment the core RNA polymerase is comprised of a β′ subunit having the amino acid sequence of SEQ ID NO: 1, a β subunit having the amino acid sequence of SEQ ID NO:2, and a pair of α subunits having the amino acid sequence of SEQ ID NO:3. More preferably, this core RNA polymerase further comprises an co subunit having the amino acid sequence of SEQ ID NO:4.

A crystal of the present invention may take a variety of forms all of which are included in the present invention. In a particular embodiment the crystal of the RNA polymerase has a space group of P4₁2₁2 and a unit cell of dimensions of a=b=201 and c=294 Å.

The present invention further includes methods of preparing a crystal of the core RNA polymerase bound to an RNAP binding partner, e.g, an RNAP inhibitor such as rifampicin. A particular method comprises first growing a core bacterial RNA polymerase crystal in a buffered solution. One such buffered solution exemplified below, contains 40-45% saturated ammonium sulfate. In one such embodiment the growing is performed by batch crystallization. In another embodiment the growing is performed by vapor diffusion. In yet another embodiment the growing is performed by microdialysis.

The crystals can be subsequently soaked in a stabilization solution, (e.g., 2 M (NH₄)₂SO₄, 0.1 M Tris-HCl, pH 8.0, and 20 mM MgCl₂) with an RNAP binding partner such as rifampicin (0.1 mM rifampicin was added in the Example below). The RNAP/RNAP-binding partner are preferably incubated in the stabilization buffer for at least twelve hours. The crystals are then prepared for cryo-crystallography by soaking the RNAP/RNAP-binding partner complex in a stabilization buffer (e.g., 2 M (NH₄)₂SO₄, 0.1 M Tris-HCl, pH 8.0, and 20 mM MgCl₂ containing 50% (w/v) sucrose) before flash freezing. As exemplified below, crystals of the Rif-RNAP complex were prepared by soaking the Rif-RNAP complex for 30 minutes in stabilization buffer prior to flash freezing in liquid nitrogen.

Alternatively, the core RNA polymerase bound to an RNAP binding partner, e.g, an RNAP inhibitor such as rifampicin, can be co-crystallized under the conditions as described above.

Preferably the crystal of the Rif-RNAP complex effectively diffracts X-rays for the determination of the atomic coordinates of the Rif-RNAP complex to a resolution of better than 5.0 Angstroms. In a preferred embodiment the crystal effectively diffracts X-rays for the determination of the atomic coordinates of the Rif-RNAP complex to a resolution of 3.5 Angstroms or better. In a particular embodiment the crystal effectively diffracts X-rays for the determination of the atomic coordinates of the Rif-RNAP complex to a resolution of 3.3 Angstroms or better.

In a particular embodiment the crystal is grown by vapor diffusion. In one such embodiment the crystal is grown by hanging-drop vapor diffusion. In another embodiment the crystal is grown by sitting-drop vapor diffusion. Standard micro and/or macro seeding may be used to obtain a crystal of X-ray quality, i.e. a crystal that will diffract to allow resolution better than 5.0 Angstroms.

Still another aspect of the present invention comprises a method of using a crystal of the present invention and/or a dataset comprising the three-dimensional coordinates obtained from the crystal in a drug screening assay.

In addition, the present invention provides three-dimensional coordinates for the Rif-RNAP complex. In a particular embodiment the coordinates are for the Rif-RNAP complex using the Thermus aquaticus core RNA polymerase as disclosed in Table 2 (in Appendix following the Sequence Listing). Thus the dataset of Table 2 below, is part of the present invention. Furthermore, the dataset of Table 2 below, in a computer readable form is also part of the present invention. In addition, methods of using such coordinates (including in computer readable form) in the drug assays and drug screens as exemplified herein, are also part of the present invention. In a particular embodiment of this type, the coordinates contained in the dataset of Table 2 below, can be used to identify potential modulators of the core RNA polymerase. In a preferred embodiment, the modulator is designed to interfere with the bacterial RNAP, but not to interfere with the human RNAP.

Accordingly, the present invention provides methods of identifying an agent or drug that can be used to treat bacterial infections. One such embodiment comprises a method of identifying an agent for use as an inhibitor of bacterial RNA polymerase using a crystal of a Rif-RNAP complex and/or a dataset comprising the three-dimensional coordinates obtained from the crystal. In a particular embodiment the three-dimensional coordinates of the Rif-RNAP complex are determined using the Thermus aquaticus core RNA polymerase. Preferably the crystal of the Rif-RNAP complex effectively diffracts X-rays for the determination of the atomic coordinates to a resolution of, or better than 3.5 Angstroms. More preferably the crystal of the Rif-RNAP complex effectively diffracts X-rays for the determination of the atomic coordinates to a resolution of, or better than 3.3 Angstroms. Preferably the selection is performed in conjunction with computer modeling.

In one embodiment the potential agent is selected by performing rational drug design with the three-dimensional coordinates determined for the crystal. As noted above, preferably the selection is performed in conjunction with computer modeling. The potential agent is then contacted with the bacterial RNA polymerase and the activity of the bacterial RNA polymerase is determined (e.g., measured). A potential agent is identified as an agent that inhibits bacterial RNA polymerase when there is a decrease in the activity determined for the bacterial RNA polymerase.

In a preferred embodiment the method further comprises preparing a supplemental crystal containing the core RNA polymerase bound to the potential agent. Preferably the supplemental crystal effectively diffracts X-rays for the determination of the atomic coordinates to a resolution of better than 5.0 Angstroms, more preferably to a resolution equal to or better than 3.5 Angstroms, and even more preferably to a resolution equal to or better than 3.3 Angstroms. The three-dimensional coordinates of the supplemental crystal are then determined with molecular replacement analysis and a second generation agent is selected by performing rational drug design with the three-dimensional coordinates determined for the supplemental crystal. Preferably the selection is performed in conjunction with computer modeling.

As should be readily apparent the three-dimensional structure of a supplemental crystal can be determined by molecular replacement analysis or multiwavelength anomalous dispersion or multiple isomorphous replacement. A candidate drug is then selected by performing rational drug design with the three-dimensional structure determined for the supplemental crystal, preferably in conjunction with computer modeling. The candidate drug can then be tested in a large number of drug screening assays using standard biochemical methodology exemplified herein.

The method can further comprise contacting the second generation agent with a eukaryotic RNA polymerase and determining (e.g., measuring) the activity of the eukaryotic RNA polymerase. A potential agent is then identified as an agent for use as an inhibitor of bacterial RNA polymerase when there is significantly less change (a factor of two or more) in the activity of the eukaryotic RNA polymerase relative to that observed for the bacterial RNA polymerase. Preferably no, or alternatively minimal change (i.e., less than 15%) in the activity of the eukaryotic RNA polymerase is determined.

The present invention further provides a method of identifying an agent that inhibits bacterial growth using the crystal of a Rif-RNAP complex or a dataset comprising the three-dimensional coordinates obtained from the crystal. In a particular embodiment the three-dimensional coordinates of the Rif-RNAP complex are determined with the Thermus aquaticus core RNA polymerase.

Preferably the Rif-RNAP complex effectively diffracts X-rays for the determination of the atomic coordinates to a resolution of, or better than 3.5 Angstroms. More preferably the Rif-RNAP complex effectively diffracts X-rays for the determination of the atomic coordinates to a resolution of, or better than 3.3 Angstroms. Preferably the selection is performed in conjunction with computer modeling.

In one embodiment the potential agent is selected by performing rational drug design with the three-dimensional coordinates determined for the crystal of the Rif-RNAP complex. As noted above, preferably the selection is performed in conjunction with computer modeling. The potential agent is contacted with and/or added to a bacterial culture and the growth of the bacterial culture is determined. A potential agent is identified as an agent that inhibits bacterial growth when there is a decrease in the growth of the bacterial culture. The method can further comprise preparing a supplemental crystal containing the core RNA polymerase formed in the presence of the potential agent. Preferably the supplemental crystal effectively diffracts X-rays for the determination of the atomic coordinates to a resolution of better than 5.0 Angstroms, more preferably to a resolution equal to or better than 3.5 Angstroms, and even more preferably to a resolution equal to or better than 3.3 Angstroms. The three-dimensional coordinates of the supplemental crystal are then determined with molecular replacement analysis and a second generation agent is selected by performing rational drug design with the three-dimensional coordinates determined for the supplemental crystal. Preferably the selection is performed in conjunction with computer modeling. The candidate drug can then be tested in a large number of drug screening assays using standard biochemical methodology exemplified herein.

In a particular embodiment the second generation agent is contacted with a eukaryotic cell and the amount of proliferation of the eukaryotic cell is determined. A potential agent is identified as an agent for inhibiting bacterial growth when there is significantly less change (a factor of two or more) in the proliferation of the eukaryotic cell relative to that observed for the bacterial cell. Preferably no, or alternatively minimal change (i.e., less than 15%) in the proliferation of the eukaryotic cell is determined.

Computer analysis may be performed with one or more of the computer programs including: QUANTA, CHARMM, INSIGHT, SYBYL, MACROMODEL and ICM [Dunbrack et al., Folding & Design, 2:27-42 (1997)]. In a further embodiment of this aspect of the invention, an initial drug screening assay is performed using the three-dimensional structure so obtained, preferably along with a docking computer program. Such computer modeling can be performed with one or more Docking programs such as DOC, GRAM and AUTO DOCK [Dunbrack et al., Folding & Design, 2:27-42 (1997)].

It should be understood that in all of the drug screening assays provided herein, a number of iterative cycles of any or all of the steps may be performed to optimize the selection. For example, assays and drug screens that monitor the activity of the RNA polymerase in the presence and/or absence of a potential modulator (or potential drug) are also included in the present invention and can be employed as the sole assay or drug screen, or more preferably as a single step in a multi-step protocol for identifying modulators of bacterial proliferation and the like.

The present invention further provides the novel agents (modulators or drugs) that are identified by a method of the present invention, along with the method of using agents (modulators or drugs) identified by a method of the present invention, for inhibiting bacterial RNA polymerase and/or bacterial proliferation.

The present invention further provides an apparatus that comprises a representation of a Rif-RNAP complex. One such apparatus is a computer that comprises the representation of the Rif-RNAP complex in computer memory. In one embodiment, the computer comprises a machine-readable data storage medium which contains data storage material that is encoded with machine-readable data which comprises the atomic coordinates obtained from a crystal of the Rif-RNAP complex. Preferably the computer comprises a machine-readable data storage medium which contains data storage material that is encoded with machine-readable data which comprises the structural coordinates of Table 2. In one embodiment, the computer comprises a machine-readable data storage medium which contains data storage material that is encoded with machine-readable data which comprises the structural coordinates obtained from a crystal of the Rif-RNAP complex. More preferably the computer further comprises a working memory for storing instructions for processing the machine-readable data, a central processing unit coupled to both the working memory and to the machine-readable data storage medium for processing the machine readable data into a three-dimensional representation of the Rif-RNAP complex. In a preferred embodiment, the computer also comprises a display that is coupled to the central-processing unit for displaying the three-dimensional representation.

Accordingly, it is a principal object of the present invention to provide a crystal containing the Rif-RNAP complex.

It is a further object of the present invention to provide the three-dimensional coordinates of the Rif-RNAP complex for the Thermus aquaticus core RNA polymerase.

It is a further object of the present invention to provide methods for the rational design of drugs that inhibit prokaryotic RNA polymerase.

It is a further object of the present invention to provide methods of identifying drugs that can modulate bacterial proliferation.

It is a further object of the present invention to provide methods for the rational design of drugs that inhibit bacterial proliferation without negatively effecting human RNA polymerase.

It is a further object of the present invention to provide methods of identifying agents that can be used to treat bacterial infections in mammals, and preferably in humans.

These and other aspects of the present invention will be better appreciated by reference to the following drawings and Detailed Description.

### BRIEF DESCRIPTION OF THE DRAWINGS

FIG. 1 depicts the rifampicin (Rif) resistant regions of the RNAP β subunit. The bar on top schematically represents the E. coli β subunit primary sequence with amino acid numbering shown directly above. Gray boxes within the schematic indicate evolutionarily conserved regions among all prokaryotic, chloroplast, archaebacterial, and eukaryotic sequences labeled A-I at the top [Allison et al., Cell 42:599-610 (1985); Sweetser et al., Proc. Natl. Acad. Sci. USA 84:1192-1196 (1987)]. Red markings indicate the four clusters where Rif^{R} mutations have been identified in E. coli [Jin and Gross, J. Molec. Biol, 202:45-58 (1988); Lisitsyn et al., Bioorg Khim 10:127-128 (1984); Lisitsyn et al., Molec. Gen. Genet., 196:173-174 (1984); Ovchinnikov et al., Molec. Gen. Genet. 190:344-348 (1983); Severinov et al., J. Biol. Chem., 268:14820-14825 (1993); Severinov et al., Molec. Gen. Genet., 244:120-126 (1994)] denoted as the N-terminal cluster (N), and clusters I, II and III (I, II, III). Directly below is a sequence alignment spanning these regions of the E. coli (E.c.), T. aquaticus (T.a.), and M. tuberculosis (M. t) RNAP β subunits. Amino, acids that are identical to E. coli are shaded dark gray, and those that are homologous (ST, RK, DE, NQ, FYWIV) are shaded light gray. Mutations that confer Rif^{R} in E. coli and M. tuberculosis are indicated directly above (for E. coli) or below (for M. tuberculosis) as follows: Δ for deletions, Ω for insertions, and colored dots for amino acid substitutions (substitutions at each position are indicated in single-amino acid code in columns above or below the positions).

Color-coding for the amino acid substitutions (for reference to subsequent figures):
(i) yellow, residues that interact directly with the bound rifampicin (see FIG. 4a-4b);
(ii) green, residues that are too far away from the rifampicin for direct interaction (see FIG. 5a-5b); and
(iii) purple, three positions that are substituted with high frequency (noted as a % immediately below the substitutions) in clinical isolates of Rif^{R} M. tuberculosis [Ramaswamy and Musser, Tubercle and Lung Disease 79:3-29 (1998)]. Below the three prokaryotic sequences is a sequence alignment of three eukaryotic sequences with shading as above. The dots indicate a gap in the alignment.

FIGS. 2a-2d show that the rifampicin inhibition of Taq RNAP. FIG. 2a depicts autoradiographs showing the radioactive RNA produced by Taq (lanes 1-7) and E. coli (lanes 8-13) RNAP holoenzymes transcribing a template containing the T7 A1 promoter and the tR2 terminator, analyzed on a 15% polyacrylamide gel and quantitated by phosphorimagery. In the absence of rifampicin (lanes 1 and 8), the major RNA products from each RNAP correspond to a trimeric abortive product (CpApU), a 105 nucleotide terminated transcript (Term), and a 127 nucleotide runoff transcript (Run off). Lanes 2-7 and 9-13 show the effects of increasing concentrations of rifampicin. FIG. 2b shows the quantitated results, where the amounts of each product (normalized to 100% for the Run off and Term transcripts in the absence of rifampicin, and for CpApU at the highest concentration of rifampicin) are plotted as a function of rifampicin concentration. FIG. 2c shows the distance between the bound rifampicin and the initiating substrate (i-site) of E. coli and Taq RNAP holoenzymes measured using chimeric Rif-nucleotide compounds as previously described [Mustaev et al., Proc. Nat. Acad. Sci. USA 91:12036-12040 (1994)]. Rif-nucleotide compounds (Rif-(CH2)n-Ap) with different linker lengths, n (indicated above each lane) were bound to RNAP, then extended in a specific transcription reaction with α-[³²P]UTP by the RNAP catalytic activity. The products were analyzed on a 23% polyacrylamide gel, visualized by autoradiography, and quantitated by phosphorimagery. FIG. 2d shows the quantitated results where the product yield (as % activity normalized to 100% at the highest level) is plotted as a function of the Rif-nucleotide linker length (n).

FIGS. 3a-3c show the Rif-RNAP co-crystal structure. FIG. 3a is a stereoview of the Rif-binding pocket of Taq core RNAP, generated using O [Jones et al., Acta Cryst, A 47:110-119 (1991)]. Carbon atoms of the RNAP β subunit are cyan or yellow (residues within 4 Å of the rifampicin), while carbon atoms of the inhibitor are orange. Oxygen atoms are red, nitrogen atoms are blue, and sulfur atoms are green. Electron density, calculated using (|Fₒ^{Rif}−Fₒ^{nat}|) coefficients is shown (orange) for the Rif only (contoured at 3.5 σ), and was computed using phases from the final refined RNAP model with the rifampicin omitted [see U.S. Ser. No. 09/396,651, Filed Sep. 15, 1999, the contents of which are hereby incorporated by reference in their entireties.] Here, “Rif” denotes the Rif-RNAP co-crystal, and “native” denotes the native core RNAP crystal. FIG. 3b shows the three-dimensional structure of Taq core RNAP in complex with rifampicin generated using GRASP [Nicholls et al., Proteins Structure, Function and Genetics 11:281-296 (1991)]. The backbone of the RNAP structure is shown as tubes, along with the color-coded transparent molecular surface (β, cyan; β′, pink; ω, white; the α-subunits are behind the RNAP and are not visible). The Mg²⁺ ion chelated at the active site is shown as a magenta sphere. The rifampicin is shown as CPK atoms (carbon, orange; oxygen, red; nitrogen, blue). FIG. 3c is the structural formula of rifampicin. Features of the structure discussed in the text are color-coded (ansa bridge, blue; napthol ring, green). The four oxygen atoms critical for rifampicin activity [Arora, Acta Crystall. B37:152-157 (1981); Arora, Molecular Pharmacology 23:133-140 (1983); Arora, J. Med. Chem. 28:1099-1102 (1985); Arora and Main, J. Antibiot. 37:178-181 (1984); Brufani et al., J. Molec. Biol. 87:409-435 (1974); Lancini and Zanichelli, In Structure-activity Relationship in Semisynthetic Antibiotics, D. Perlaman, ed. (Academic Press), pp. 531-600 (1977); Sensi et al., Rev. Infect. Dis., 5 Supp.3:402-406 (1983)] are shaded with red circles.

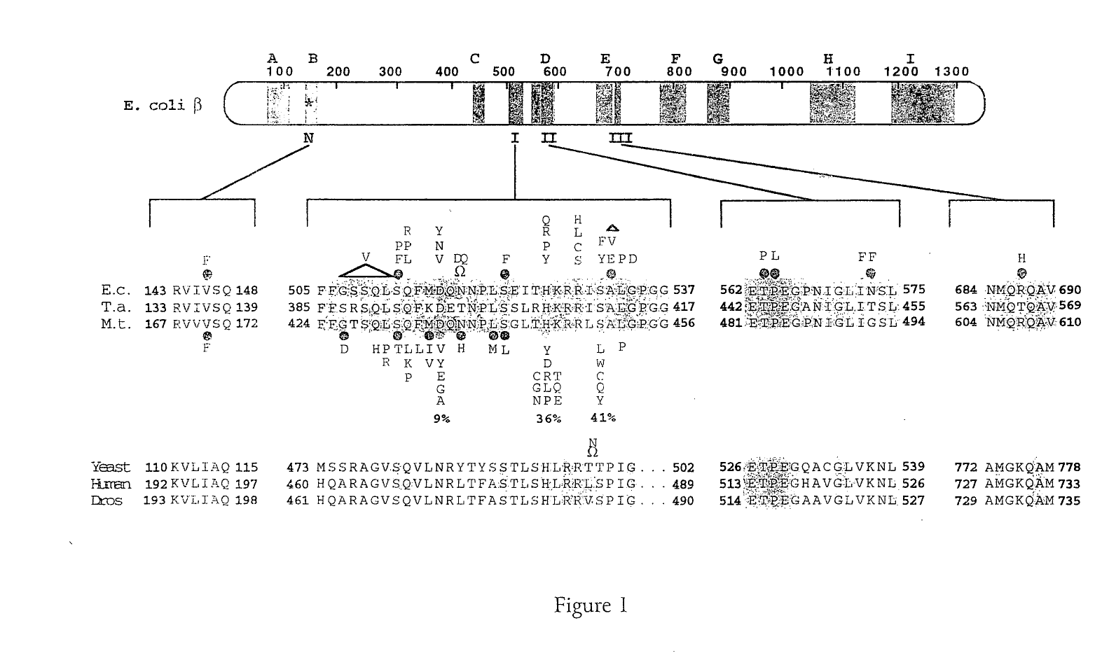

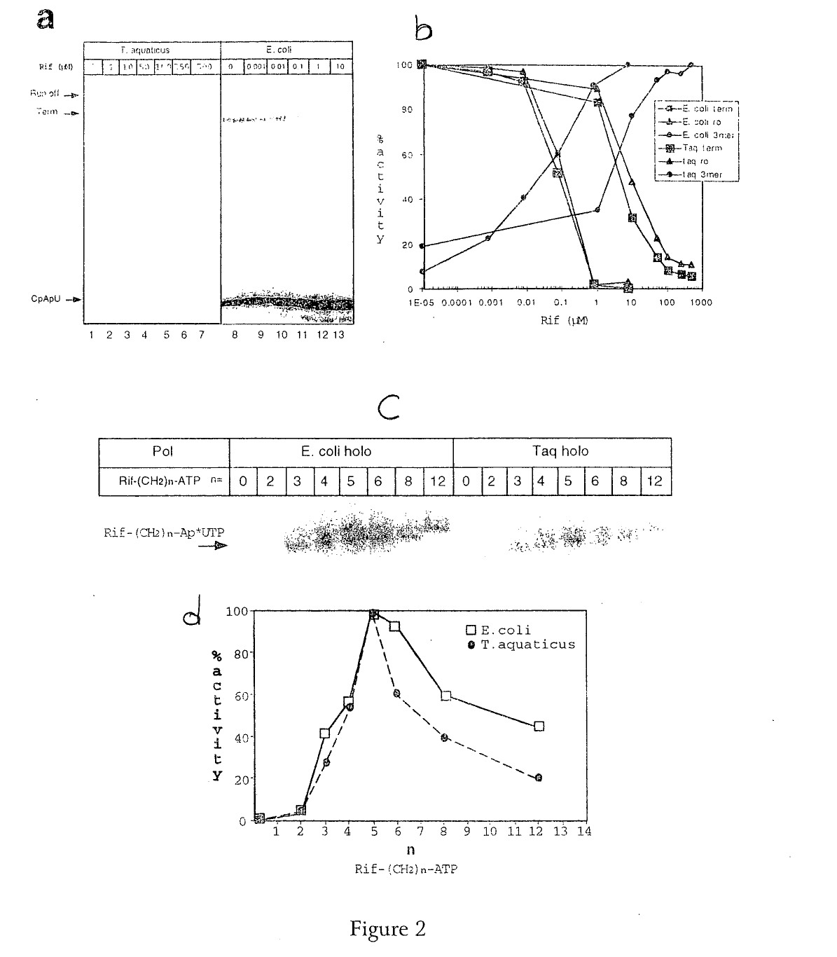

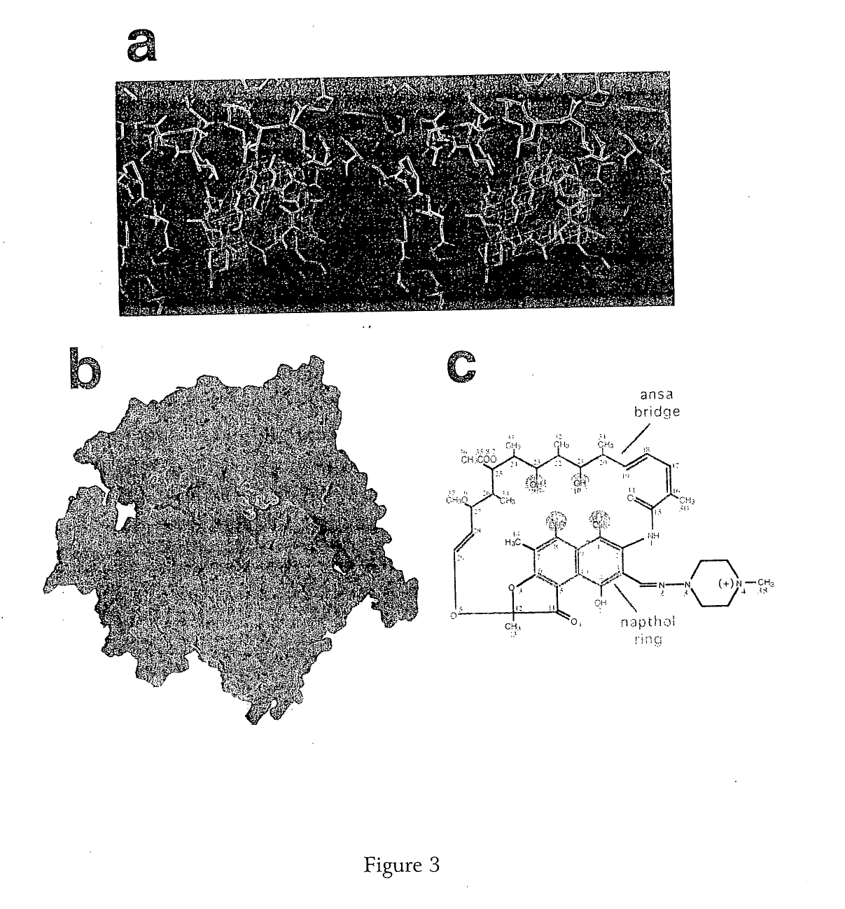

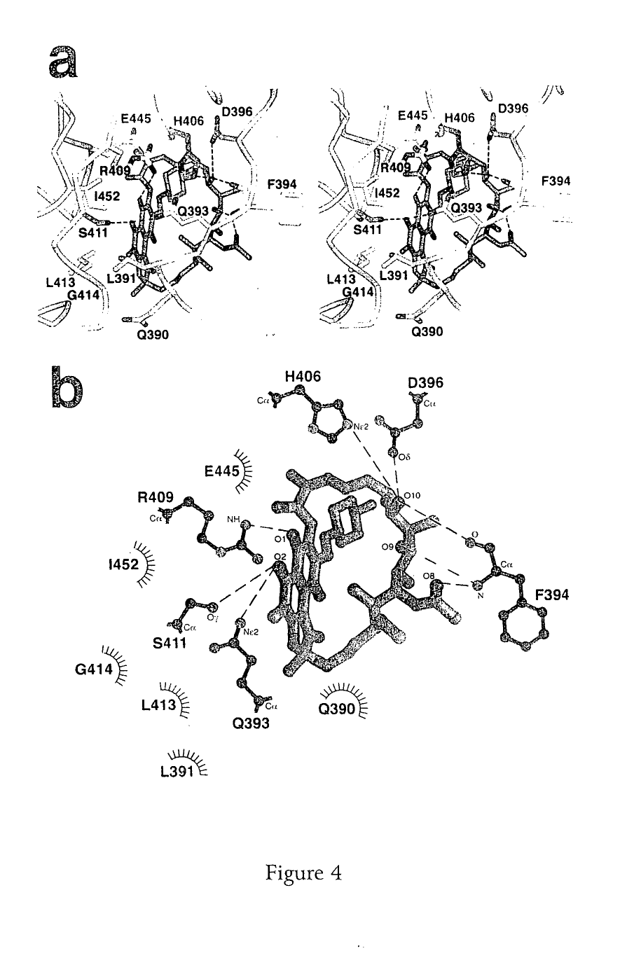

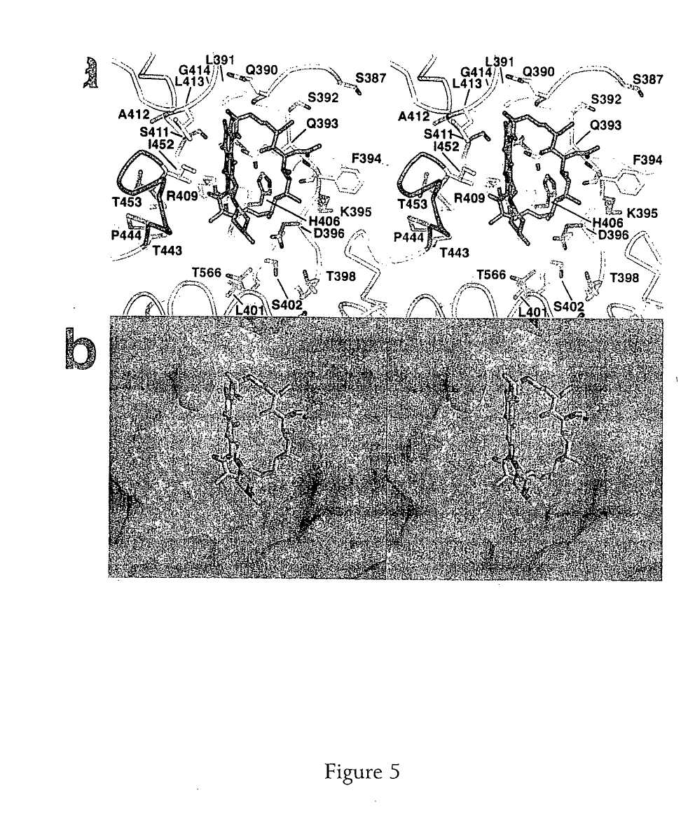

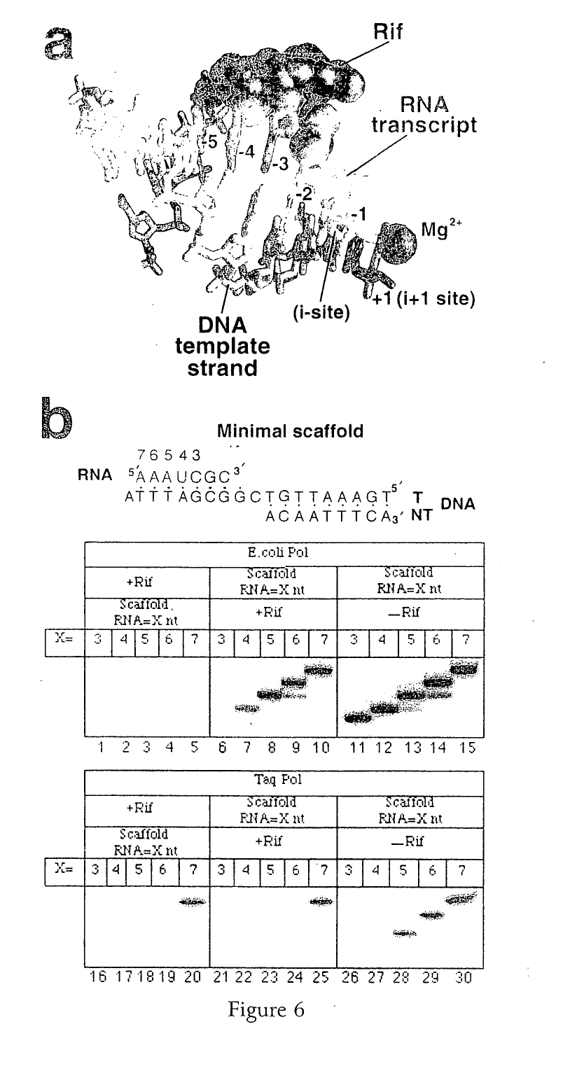

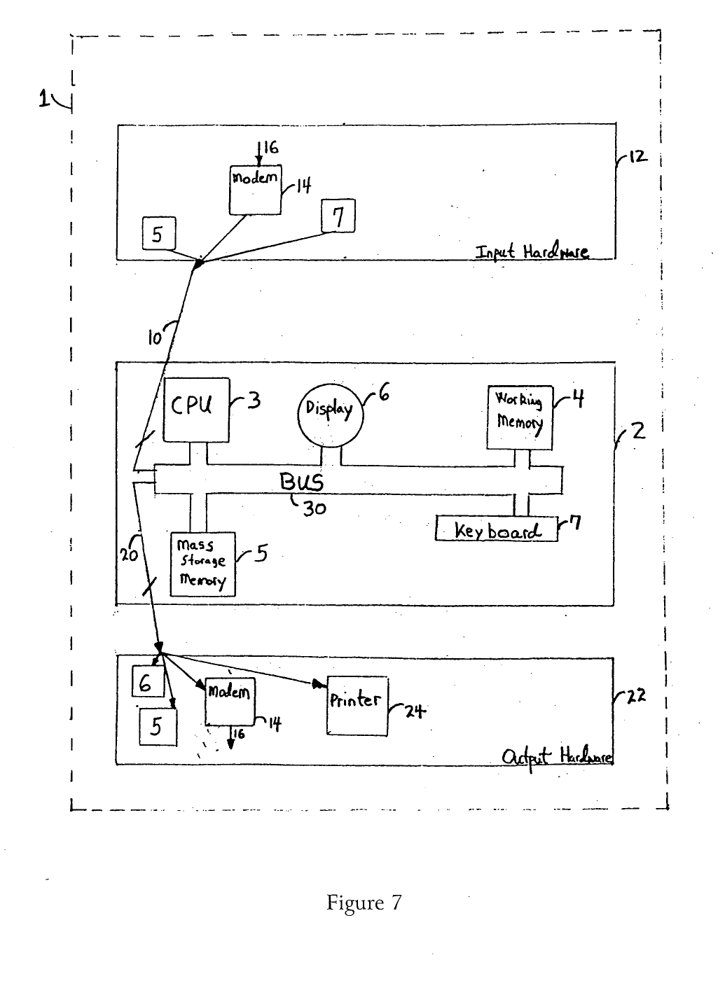

FIGS. 4a-4b depict the detailed interactions of rifampicin with RNAP. FIG. 4a is a stereoview of the Taq RNAP Rif binding pocket complexed with rifampicin, generated using RIBBONS [Carson, J. Appl. Crystall., 24:958-961 (1991)], showing residues that interact directly with the inhibitor. The backbone of the β subunit is shown as a cyan ribbon. Side chains (and backbone atoms of F394) of residues within 4 Å of rifampicin are shown. Carbon atoms are orange (Rif), magenta (three residues substituted in M. tuberculosis Rif^{R} clinical isolates with high frequency, see FIG. 1), or yellow; oxygen atoms are red; nitrogen atoms are blue. The view is from above the β subunit, looking through β to the rifampicin, but with obscuring parts of β removed. Potential hydrogen bonds between protein atoms and rifampicin are shown as dashed lines. FIG. 4b shows a schematic drawing of RNAP β subunit interactions with rifampicin, modified from LIGPLOT [Wallace et al., Protein Engineering 8:127-134 (1995)]. Residues forming van-der-Waals interactions are indicated: those participating in hydrogen bonds are shown in a ball-and-stick representation, with hydrogen bonds depicted as dashed lines, carbon atoms of the protein are black, while carbon atoms of rifampicin are orange. Oxygen atoms are red and nitrogen atoms are blue.

FIGS. 5a-5b show the rifampicin binding pocket and Rif^{R} mutants as stereoviews of the Taq RNAP Rif binding pocket complexed with rifampicin. The view is the same in FIGS. 5a and 5b and is rotated approximately 180°about the horizontal axis from the view of FIG. 4a. This view is from the middle of the main RNAP channel, looking towards the rifampicin, with the β subunit behind. FIG. 5a shows the backbone of the β subunit as a cyan ribbon, but with a highly conserved segment of region D (443-451, see text) colored red. Side chains (and backbone atoms of F394) of residues where substitutions confer Rif^{R} (see FIG. 1) are shown. Carbon atoms are orange (Rif), magenta (three residues substituted in M. tuberculosis Rif^{R} clinical isolates with high frequency, see FIG. 1), yellow (other residues that interact directly with rifampicin, as in FIG. 4), or green (all other Rif^{R} positions). Oxygen atoms are colored red; nitrogen atoms are blue. The depiction was generated using RIBBONS [Carson, J. Appl. Crystall., 24:958-961 (1991)]. The β subunit is shown in FIG. 5b as a cyan molecular surface, with a highly conserved segment of region D colored red, and surface exposed Rif^{R} positions colored yellow (within 4 Å of the Rif) or green. The depiction was generated using GRASP [Nicholls et al., Proteins Structure, Function and Genetics 11:281-296 (1991)].

FIGS. 6a and 6b show the mechanism of RNAP inhibition by rifampicin. The RNAP active site Mg²⁺ (magenta sphere) and the 9-basepair RNA/DNA hybrid (from +1 to −8) from a model of the ternary elongation complex [Korzheva et al., Science 289:619-625 (2000)] are shown in FIG. 6a. The RNAP itself and the rest of the nucleic acids are omitted for clarity. The incoming nucleotide substrate at the +1 position is colored green, the −1 and −2 positions, which can be accommodated in the presence of rifampicin, are colored yellow. The RNA further upstream (−3 to −8), which cannot be accommodated in the presence of rifampicin is colored pink. The template strand of the DNA is colored grey. Also shown is a CPK representation of rifampicin as it would be positioned in its binding site on the β subunit (carbon atoms, orange; oxygen, red; nitrogen, blue). The rifampicin is partially transparent, illustrating the RNA nucleotides at −3 to −5 that sterically clash. This depiction was generated using GRASP [Nicholls et al., Proteins Structure, Function and Genetics 11:281-296 (1991)]. The structure of the minimal scaffold systems with RNA lengths from 3-7 nucleotides (labeled above the RNA chain) are shown in FIG. 6b [Korzheva et al., Science 289:619-625 (2000)]. The results are presented below as autoradiographs of the radioactive RNAs produced by E. coli (lanes 1-15) or Taq (lanes 16-30) core RNAPs transcribing the minimal scaffolds with the indicated lengths of RNA (‘X=’) and analyzed on a 23% polyacrylamide gel. Lanes 1-10 and 16-25 demonstrate the effect of rifampicin inhibition on transcription when it was bound by RNAP either before (lanes 1-5 and 16-20) or after (lanes 6-10 and lanes 21-25) addition of the scaffold. Lanes 11-15 and 26-30 show elongation of the same scaffolds in the absence of rifampicin. The RNA with the critical length of 3 nucleotides which cannot be elongated by E. coli RNAP in the presence of rifampicin regardless of the order of rifampicin and scaffold addition (lanes 1,6) is colored red. The RNAs of 4-7 nucleotides (colored green) were extended by E. coli RNAP when added before rifampicin (lanes 6-10).

FIG. 7 depicts a schematic of a computer comprising a central processing unit (“CPU”), a working memory, a mass storage memory, a display terminal, and a keyboard that are interconnected by a conventional bidirectional system bus. The computer can be used to display and manipulate the structural data of the present invention.

### DETAILED DESCRIPTION OF THE INVENTION

The present invention provides crystals of a bacterial core RNA polymerase bound to an inhibitor. The present invention further provides the structural coordinates for a bacterial core RNA polymerase bound to rifampicin (Rif-RNAP complex) and methods of using such structural coordinates in drug assays. More particularly, the present invention provides the structural coordinates for the Rif-RNAP complex with the Thermus aquaticus core RNA polymerase (see Table 2 in Appendix following the Sequence Listing).

Rifampicin (Rif) is one of the most potent and broad-spectrum antibiotics against bacterial pathogens and is a key component of anti-tuberculosis therapy, stemming from its inhibition of the bacterial RNA polymerase (RNAP). The X-ray crystal structure of Thermus aquaticus core RNA polymerase reveals a ‘crab-claw’ shaped molecule with a 27 Å wide internal channel [see U.S. Ser. No. 09/396,651, Filed Sep. 15, 1999, the contents of which are hereby incorporated by reference in their entireties]. As disclosed herein, rifampicin binds in a pocket of the RNAP subunit deep within the DNA/RNA channel, but more than 12 Å away from the active site the crystal structure of Thermus aquaticus core RNAP complexed with rifampicin. The structure, combined with biochemical results disclosed herein, explains the effects of rifampicin on RNAP function and indicates that the inhibitor acts by directly blocking the path of the elongating RNA when the transcript becomes 2 to 3 nucleotides in length.

The three-dimensional structure disclosed herein demonstrates that rifampicin binds the Taq core RNAP with a close complementary fit in a pocket between two structural domains of the RNAP β subunit. Only small, local conformational changes of both the inhibitor and the protein were observed. The binding site is deep within the main RNAP channel, but the closest approach of the inhibitor to the RNAP active site Mg²⁺ is more than 12 Å (FIG. 3b, below). The Rif binding pocket is surrounded by the 23 known positions where amino acid substitutions confer Rif^{R} (FIG. 5, below). Twelve of these residues are close enough to interact directly with the rifampicin (FIGS. 4a-4b, below). Predominant are van-der-Waals interactions with hydrophobic side-chains near the napthol ring of rifampicin, and potential hydrogen bond interactions with 5 polar groups of rifampicin (2 on the napthol ring, and 3 on the ansa bridge), 4 of which have been shown to be essential for rifampicin activity. The remaining known Rif^{R} mutants are one layer removed from the rifampicin itself, and are likely to affect rifampicin binding through small structural distortions of the Rif binding pocket.

Therefore the structure disclosed herein explains the effects of rifampicin on RNAP function determined from detailed biochemical and kinetic studies. In combination with a model of the ternary transcription complex, the structure indicates that the predominant effect of rifampicin is to directly block the path of the elongating RNA transcript at the 5′-end when the transcript becomes either 2 or 3 nucleotides in length, depending on the 5′-phosphorylation state of the 5′-nucleotide (FIGS. 6a-6b, below). In this view, rifampicin binds the Rif binding site of the RNAP holoenzyme either before or after the binding of the DNA template and formation of the open complex. Indeed, the binding of the DNA template and the formation of the open complex are not affected by the presence of rifampicin. However, rifampicin has its effect after the nucleotide substrates binds their sites in the RNAP active site. Thus the initiating nucleotide substrate binds the RNAP i-site with a small, approximately 2-fold increase in the apparent Km due to the presence of rifampicin, while the second nucleotide binds in the i+1 site with little notice of the rifampicin. More or less normally, the RNAP then catalyzes the formation of a phosphodiester bond between the two nucleotides. If the initiating nucleoside bears a 5′-triphosphate, the subsequent translocation of the RNAP attempts to move the 2-nucleotide RNA transcript upstream such that the i+1 nucleotide occupies the i-site (−1 position), and the i-site nucleotide moves into the −2 position (FIG. 6a, below). The movement of the 5′-nucleotide into the −2 position, however, results in a severe steric clash with the rifampicin. The molecular details of the ensuing events are unclear, but in the end the RNAP remains at the same template position, the 2-nucleotide transcript is released, and the futile cycle begins again. If the 5′-nucleoside contains a di- or a mono-phosphate at its 5′-end (or if it's unphosphorylated), then after the synthesis of the first phosphodiester bond, the RNAP can translocate normally and the steric clash of the transcript with the bound rifampicin occurs during the translocation of the 3-nucleotide transcript following the synthesis of the second phosphodiester bond.

The present invention exploits the structural information described herein, including the structural coordinates disclosed in Table 2, and provides methods of identifying agents or drugs that can be used to control the proliferation of bacteria, e.g., for use as treatments for bacterial infections.

Therefore, if appearing herein, the following terms shall have the definitions set out below:

As used herein the term “core RNA polymerase” minimally comprises the subunit composition of α₂ββ′ which is evolutionarily conserved from bacteria to man. Preferably the core RNA polymerase further comprises the ω subunit. The three-dimensional structure of the Thermus aquaticus core RNA polymerase was disclosed in U.S. Ser. No. 09/396,651, Filed Sep. 15, 1999, the contents of which are hereby incorporated by reference in their entireties.

As used herein “Rif-RNAP” is used interchangeably with the “Rif-RNAP complex” and comprises the binding complex of rifampicin with the core RNA polymerase as disclosed in the Example below. The structural coordinates for a crystal of Rif-RNAP are listed in Table 2 (in Appendix following the Sequence Listing).

As used herein an “RNAP binding partner” is a small organic molecule that binds to RNAP. Preferably the RNAP binding partner is an inhibitor of the catalytic and/or the transcriptional activity of RNAP. Rifampicin is a particular binding partner of RNAP that is exemplified below.

As used herein, the “transcriptional activity of RNAP” includes the ability of RNAP to carry out the elongation of the RNA transcript during transcription. Thus, whereas the catalytic activity of RNAP includes the binding of the enzyme to the nucleotide substrates and the subsequent formation of the phosphodiester bond between the two substrates, the transcriptional activity includes the RNAP dependent elongation of the RNA transcript at the 5′-end.

As used herein an “active RNA polymerase” is an RNA polymerase that minimally contains a pair of α subunits, a β′ subunit, and a β subunit; or fragments thereof, but still retains at least 25% of the catalytic and/or transcriptional activity of the core RNA polymerase made up of the full length α, β′, and β subunits. Thus active RNA polymerases can comprise fragments of the α subunit and/or β′ subunit and/or 1 subunit.

As used-herein a “small organic molecule” is an organic compound [or organic compound complexed with an inorganic compound (e.g., metal)] that has a molecular weight of less than 3 Kd.

As used herein the term “about” means within 10 to 15%, preferably within 5 to 10%. For example an amino acid sequence that contains about 60 amino acid residues can contain between 51 to 69 amino acid residues, more preferably 57 to 63 amino acid residues.

As used herein a polypeptide or peptide “consisting essentially of” or that “consists essentially of” a specified amino acid sequence is a polypeptide or peptide that retains the general characteristics, e.g., activity of the polypeptide or peptide having the specified amino acid sequence and is otherwise identical to that protein in amino acid sequence except it consists of plus or minus 10% or fewer, preferably plus or minus 5% or fewer, and more preferably plus or minus 2.5% or fewer amino acid residues.

As used herein, and unless otherwise specified, the terms “agent”, “potential drug”, “test compound” or “potential compound” are used interchangeably, and refer to chemicals which potentially have a use as a modulator (and preferably as an inhibitor) of bacterial RNA polymerase. More preferably, an agent is a drug that can be used to treat and/or prevent bacterial infection. Therefore, such “agents”, “potential drugs”, and “potential compounds” may be used, as described herein, in drug assays and drug screens and the like.

Nucleic Acids Encoding Subunits of Bacterial RNA polymerases

The present invention contemplates isolation of nucleic acids encoding a subunit of an RNA polymerase including a full length, i.e., naturally occurring form of the RNA polymerase from any prokaryotic source, preferably a thermophilic bacterial source. The present invention further provides for subsequent modification of the nucleic acid to generate a fragment or modification of the subunit that can still be used to form a core RNA polymerase that will crystallize.

In accordance with the present invention there may be employed conventional molecular biology, microbiology, and recombinant DNA techniques within the skill of the art. Such techniques are explained fully in the literature [see, e.g., Sambrook and Russell Molecular Cloning: A Laboratory Manual, Third Edition (2001) Vols. I-III, Cold Spring Harbor Laboratory Press, Cold Spring Harbor, N.Y. (herein “Sambrook and Russell, 2001”)].

Therefore, if appearing herein, the following terms shall have the definitions set out below.

As used herein, the term “gene” refers to an assembly of nucleotides that encode a polypeptide, and includes cDNA and genomic DNA nucleic acids.

A “vector” is a replicon, such as plasmid, phage or cosmid, to which another DNA segment may be attached so as to bring about the replication of the attached segment.

A “replicon” is any genetic element (e.g., plasmid, chromosome, virus) that functions as an autonomous unit of DNA replication in vivo, i.e., capable of replication under its own control.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous DNA” refers to DNA not naturally located in the cell, or in a chromosomal site of the cell. Preferably, the heterologous DNA includes a gene foreign to the cell.

A “heterologous nucleotide sequence” as used herein is a nucleotide sequence that is added to a nucleotide sequence of the present invention by recombinant methods to form a nucleic acid which is not naturally formed in nature. Such nucleic acids can encode chimeric and/or fusion proteins. Thus the heterologous nucleotide sequence can encode peptides and/or proteins which contain regulatory and/or structural properties. In another such embodiment the heterologous nucleotide can encode a protein or peptide that functions as a means of detecting the protein or peptide encoded by the nucleotide sequence of the present invention after the recombinant nucleic acid is expressed. In still another embodiment the heterologous nucleotide can function as a means of detecting a nucleotide sequence of the present invention. A heterologous nucleotide sequence can comprise non-coding sequences including restriction sites, regulatory sites, promoters and the like.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength [see Sambrook and Russell, 2001, supra]. The conditions of temperature and ionic strength determine the “stringency” of the hybridization. For preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5x SSC, 0.5% SDS). Moderate stringency hybridization conditions correspond to a higher Tₘ, e.g., 40% formamide, with 5x or 6x SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived [see Sambrook and Russell, 2001, supra]. For hybridization with shorter nucleic acids, i.e., oligonucleotides, the position of mismatches becomes more important, and the length of the oligonucleotide determines its specificity [see Sambrook and Russell, 2001, supra]. Preferably a minimum length for a hybridizable nucleic acid is at least about 12 nucleotides; preferably at least about 18 nucleotides; and more preferably the length is at least about 27 nucleotides; and most preferably 36 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a particular embodiment the hybridization and wash conditions are identical,

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control 25: sequences in a cell when RNA polymerase transcribes the coding sequence into mRNA, which may then be trans-RNA spliced and translated into the protein encoded by the coding sequence.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) [Reeck et al., Cell, 50:667 (1987)].

Accordingly, the term “sequence similarity” in all its grammatical forms refers to the degree of identity or correspondence between nucleic acid or amino acid sequences of proteins that do not share a common evolutionary origin [see Reeck et al., 1987, supra]. However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Sambrook and Russell, 2001, supra.

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program with the default parameters.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

A gene encoding an RNA polymerase, including genomic DNA or cDNA, can be isolated from any source, particularly from a thermophilic bacterial source. In view and in conjunction with the present teachings, methods well known in the art, as described above can be used for obtaining the genes encoding an RNA polymerase from any source [see, e.g., Sambrook and Russell, 2001, supra].

Accordingly, any cell potentially can serve as the nucleic acid source for the molecular cloning of a gene encoding RNA polymerase. The DNA may be obtained by standard procedures known in the art from cloned DNA (e.g., a DNA “library”), and preferably is obtained from a cDNA library, by cDNA cloning, or by the cloning of genomic DNA, or fragments thereof, purified from the desired cell [See, for example, Sambrook and Russell, 2001, supra]. Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

The present invention also relates to cloning vectors containing genes encoding analogs and derivatives of RNA polymerase including and fragments of the various subunits, that can form active forms of RNA polymerase. Included are homologs of RNA polymerase and fragments thereof, from other species. Therefore the production and use of derivatives and analogs related to RNA polymerase are within the scope of the present invention.

RNA polymerase derivatives can be made by altering encoding nucleic acid sequences by substitutions, additions or deletions including to provide for functionally equivalent molecules. Preferably, derivatives are made that are capable of forming crystals with ligands (e.g., inhibitors) of the RNA polymerase with the crystals capable of effectively diffracting X-rays for the determination of the atomic coordinates of the protein-ligand complex to a resolution of better than 5.0 Angstroms, preferably to a resolution equal to or better than 3.5 Angstroms.

Due to the degeneracy of nucleotide coding sequences, other DNA sequences which encode substantially the same amino acid sequence as a RNA polymerase gene may be used in the practice of the present invention. These include but are not limited to allelic genes, homologous genes from other species, and nucleotide sequences comprising all or portions of RNA polymerase genes which are altered by the substitution of different codons that encode the same amino acid residue within the sequence, thus producing a silent change. Likewise, the RNA polymerase derivatives of the invention include, but are not limited to, those containing, as a primary amino acid sequence, all or part of the amino acid sequence of a RNA polymerase including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced at a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

The genes encoding RNA polymerase derivatives and analogs of the invention can be produced by various methods known in the art. The manipulations which result in their production can occur at the gene or protein level. For example, the cloned RNA polymerase gene sequence can be modified by any of numerous strategies known in the art [Sambrook and Russell, 2001, supra]. The sequence can be cleaved at appropriate sites with restriction endonuclease(s), followed by further enzymatic modification if desired, isolated, and ligated in vitro. In the production of the gene encoding a derivative or analog of RNA polymerase, care should be taken to ensure that the modified gene remains within the same translational reading frame as the RNA polymerase gene, uninterrupted by translational stop signals, in the gene region where the desired activity is encoded.

Additionally, the RNA polymerase-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Preferably, such mutations enhance the functional activity and crystallization properties of the mutated RNA polymerase gene product. Any technique for mutagenesis known in the art can be used, including but not limited to, in vitro site-directed mutagenesis [Hutchinson, et al., J. Biol. Chem. 253:6551 (1978); Zoller and Smith, DNA 3:479-488 (1984); Oliphant et al., Gene 44:177 (1986); Hutchinson et al., Proc. Natl. Acad. Sci. U.S.A. 83:710 (1986)], use of TAB® linkers (Pharmacia), etc. PCR techniques are preferred for site directed mutagenesis [see Higuchi, “Using PCR to Engineer DNA”, in PCR Technology: Principles and Applications for DNA Amplification, H. Erlich, ed., Stockton Press, Chapter 6, pp. 61-70 (1989)].

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2μ plasmid.

In an alternative method, the desired gene may be identified and isolated after insertion into a suitable cloning vector in a “shot gun” approach. Enrichment for the desired gene, for example, by size fractionation, can be done before insertion into the cloning vector.

### Expression of RNA Polymerase

The nucleotide sequence coding for RNA polymerase, a fragment of RNA polymerase or a derivative or analog thereof, including a functionally active derivative, such as a chimeric protein, thereof, can be inserted into an appropriate expression vector, i.e., a vector which contains the necessary elements for the transcription and translation of the inserted protein-coding sequence. Such elements are termed herein a “promoter.” Thus, the nucleic acid encoding a RNA polymerase of the invention or a fragment thereof is operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by the native gene encoding RNA polymerase and/or its flanking regions.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant RNA polymerase protein of the invention, or RNA polymerase fragment, derivative, chimeric construct, or analog thereof, may be expressed chromosomally, after integration of the coding sequence by recombination. In this regard, any of a number of amplification systems may be used to achieve high levels of stable gene expression [See Sambrook and Russell, 2001, supra].

The cell containing the recombinant vector comprising the nucleic acid encoding RNA polymerase is cultured in an appropriate cell culture medium under conditions that provide for expression of RNA polymerase by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of RNA polymerase may be controlled by any promoter/enhancer element known in the art, but these regulatory elements must be functional in the host selected for expression. Promoters that may be used to control RNA polymerase gene expression are well known in the art including prokaryotic expression vectors such as the β-lactamase promoter [Villa-Kamaroff, et al., Proc. Natl. Acad. Sci. U.S.A., 75:3727-3731 (1978)], or the tac promoter [DeBoer, et al., Proc. Natl. Acad. Sci. U.S.A., 80:21-25 (1983)].

Expression vectors containing a nucleic acid encoding an RNA polymerase of the invention can be identified by a number of means including four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding RNA polymerase is inserted within the “selection marker” gene sequence of the vector, recombinants containing the RNA polymerase insert can be identified by the absence of the selection marker gene function. In the fourth approach, recombinant expression vectors can be identified by assaying for the activity, biochemical, or immunological characteristics of the RNA polymerase expressed by the recombinant, provided that the expressed protein assumes a functionally active conformation.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX [Smith et al., Gene, 67:31-40 (1988)], pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamH1 cloning site; Summers), pVL1393 (BamH1, SmaI, XbaI, EcoR1, NotI, XmaIII, BglIII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedrin initiation codon; Invitrogen(195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220)) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF1S (EcoRI, PstI, SalI, AccI, HindII, SbaI, BamH1, and Hpa cloning site, TK or XPRT selection).

Yeast expression systems can also be used according to the invention to express the bacterial RNA polymerase. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter [see, e.g., Wu et al., J. Biol. Chem., 267:963-967 (1992); Wu and Wu, J. Biol. Chem., 263:14621-14624 (1988); Hartmut et al., Canadian Patent Application No. 2,012,311, filed Mar. 15, 1990).

### Peptide Synthesis

Synthetic polypeptides, prepared using the well known techniques of solid phase, liquid phase, or peptide condensation techniques, or any combination thereof, can include natural and unnatural amino acids. Amino acids used for peptide synthesis may be standard Boc (N^{α}-amino protected N^{α}-t-butyloxycarbonyl) amino acid resin with the standard deprotecting, neutralization, coupling and wash protocols of the original solid phase procedure of Merrifield [J. Am. Chem. Soc., 85:2149-2154 (1963)], or the base-labile N^{α}-amino protected 9-fluorenylmethoxycarbonyl (Fmoc) amino acids first described by Carpino and Han [J. Org. Chem., 37:3403-3409 (1972)]. Both Fmoc and Boc N^{α}-amino protected amino acids can be obtained from Fluka, Bachem, Advanced Chemtech, Sigma, Cambridge Research Biochemical, Bachem, or Peninsula Labs or other chemical companies familiar to those who practice this art. In addition, the method of the invention can be used with other N^{α}-protecting groups that are familiar to those skilled in this art. Solid phase peptide synthesis may be accomplished by techniques familiar to those in the art and provided, [e.g., Stewart and Young, Solid Phase Synthesis, Second Edition, Pierce Chemical Co., Rockford, Ill. (1984); Fields and Noble, Int. J. Pept. Protein Res. 35:161-214 (1990)], or using automated synthesizers, such as sold by ABS. Thus, polypeptides of the invention may comprise D-amino acids, a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, α-helices, β turns, β sheets, γ-turns, and cyclic peptides can be generated.

### Isolation and Crystallization of the Bacterial RNA Polymerase

The present invention provides a crystal of the Rif-RNAP complex that can be effectively diffract X-rays for the determination of the atomic coordinates of the Rif-RNAP to a resolution of better than 5.0 Angstroms and preferably to a resolution equal to or better than 3.5 Angstroms. The RNA polymerase can be expressed either as described above or as described in U.S. Ser. No. 09/396,651, Filed Sep. 15, 1999, the contents of which are hereby incorporated by reference in their entireties. Of course, the specific Rif-RNAP complex provided herein serves only as example, since the crystallization process can tolerate a broad range of active RNA polymerases and inhibitors. Therefore, any person with skill in the art of protein crystallization having the present teachings and without undue experimentation could crystallize a large number of alternative forms of the RNA polymerases from a variety of RNA polymerase fragments, or alternatively using a full length RNA polymerase from a related source and then allow the RNA polymerase to bind rifampicin and/or other RNAP binding partners (e.g., inhibitors) as described below. As mentioned above, an RNA polymerase having conservative substitutions in its amino acid sequence are also included in the invention, including a selenomethionine substituted form.

Crystals of the RNA polymerase can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used.

The crystals of the RNA polymerase can be grown alone or co-crystallized with a binding partner such as rifampicin. If the crystals are grown alone they can be subsequently soaked in a stabilization buffer with an RNAP binding partner such as rifampicin (0.1 mM rifampicin was added in the Example below). The RNAP/RNAP-binding partner are preferably incubated in the stabilization buffer for at least twelve hours. An exemplary stabilization buffer contains between 1.7-2.3 M (NH₄)₂SO₄, 0.02-1 M Tris-HCl, pH 6.5-8.5, and approximately 20 mM MgCl₂.

The crystals are then prepared for cryo-crystallography by soaking the RNAP/RNAP-binding partner complex in a stabilization buffer (e.g., 2 M (NH₄)₂SO₄, 0.1 M Tris-HCl, pH 8.0, and 20 mM MgCl₂ containing 50% (w/v) sucrose) before flash freezing.

Aside from the methodology exemplified below, alternative methods may also be used to characterize the crystals. For example, crystals can be characterized by using X-rays produced in a conventional source (such as a sealed tube or a rotating anode) or using a synchrotron source. Methods of characterization include, but are not limited to, precision photography, oscillation photography and diffractometer data collection. Selenium-Methionine may be used, or alternatively a mercury derivative dataset (e.g., using PCMB) could be used in place of the selenium-methionine derivatization.

Structural determinations can be performed by calculating Patterson maps using PHASES [Furey and Swaminathan, Methods Enzymol., 277:590-620 (1997)] for the ethyl-HgCl₂ and Ta₆Br₁₄ derivatives and using the Pb-derivative as native, for example. In the Example below, the native core RNAP structure [Zhang et al., Cell 98:811-824 (1999); U.S. Ser. No. 09/396,651, Filed Sep. 15, 1999, the contents of which are hereby incorporated by reference in their entireties] was used as a starting model for rigid body refinement and positional refinement against the observed amplitudes from the Rif-RNAP complex crystal (Fₒ^{Rif}:) using CNS [Adams et al., Proc. Natl. Acad. Sci. USA, 94:5018-5023 (1997)], yielding an initial R-factor of 0.354 (R_{free}=0.41, where the same set of reflections was set aside as was used for the R_{free} determination of the native structure) for data from 100-3.2 Å resolution. An initial Fourier difference map, calculated using |Fₒ^{Rif}−Fₒ^{nat}| amplitude coefficients and using phases calculated from the native core RNAP structure (φ^{nat}) clearly revealed density for the rifampicin molecule (FIG. 3a). Multiple rounds of manual rebuilding against (2|Fₒ|−|F_{c}|) maps using 0 [Jones et al., Acta Cryst, A 47:110-119 (1991)], and refinement using CNS [Adams et al., Proc. Natl. Acad. Sci. USA, 94:5018-5023 (1997)] resulted in the current model (Table 1). At later stages of the refinement, the rifampicin X-ray crystal structure [Brufani et al., J. Molec. Biol. 87:409-435 (1974)] was placed into the difference density. Included in the model is the recently determined sequence of the Taq 6) subunit modeled earlier as a polyalanine chain [Zhang et al., Cell 98:811-824 (1999); U.S. Ser. No. 09/396,651, Filed Sep. 15, 1999, the contents of which are hereby incorporated by reference in their entireties]. Absent from the model is a 300 amino acid, non-conserved domain inserted between conserved regions A and B of the β′ subunit [Zhang et al., Cell 98:811-824 (1999); U.S. Ser. No. 09/396,651, Filed Sep. 15, 1999, the contents of which are hereby incorporated by reference in their entireties].

### Protein-structure Based Design of Inhibitors of Bacterial RNA Polymerase

Once the three-dimensional structure of a crystal comprising a Rif-RNAP complex is determined, (e.g., see the coordinates in Table 2 below, in Appendix following the Sequence Listing) a potential modulator of RNA Polymerase, can be examined through the use of computer modeling using a docking program such as GRAM, DOCK, or AUTODOCK [Dunbrack et al., Folding & Design, 2:2742 (1997)], to identify potential modulators of the RNA Polymerase. This procedure can include computer fitting of potential modulators to the RNA Polymerase to ascertain how well the shape and the chemical structure of the potential modulator will bind to either the individual bound subunits or to the RNA Polymerase [Bugg et al., Scientific American, Dec.:92-98 (1993); West et al., TIPS, 16:67-74 (1995)]. Computer programs can also be employed to estimate the attraction, repulsion, and steric hindrance of the subunits with a modulator/inhibitor (e.g., the RNA Polymerase and a potential stabilizer).

Indeed, the shape of RNA polymerase resembles a crab-claw, with an internal groove or channel running along the full-length (between the claws). The molecule is about 150 Å long (from the back to the tips of the claws), 115 Å tall, and 110 Å wide (along the direction of the channel). The channel has many internal features, but the overall width is about 27 Å [see, U.S. Ser. No. 09/396,651, Filed Sep. 15, 1999, the contents of which are hereby incorporated by reference in their entireties].

As disclosed herein the three-dimensional structure demonstrates that rifampicin binds the Taq core RNAP with a close complementary fit in a pocket between two structural domains of the RNAP β subunit. Only small, local conformational changes of both the inhibitor and the protein is observed. The binding site is deep within the main RNAP channel, but the closest approach of the inhibitor to the RNAP active site Mg²⁺ is more than 12 Å.

Importantly, the structural information disclosed herein demonstrates that rifampicin inhibits RNA polymerase by physically blocking transcription elongation. This is in direct contrast with the modus operandi of a classical enzyme inhibitor which generally binds to the catalytic center or with a key transition state intermediate. Therefore, the effect of rifampicin depends only on its ability to bind tightly to a relatively non-conserved part of the structure, thereby disrupting a critical RNAP function. Thus, the structural information disclosed herein provides the impetus to investigate the binding of other unrelated small molecules to any of a variety of sites within the RNAP channel, which could also block transcription elongation. A preferred site is one that is critical for the transcriptional activity of bacterial RNA polymerase, but one that is not required by the corresponding mammalian enzyme.

Towards this end, generally the tighter the fit, the lower the steric hindrances, and the greater the attractive forces, the more potent the potential modulator (e.g., an inhibitor) since these properties are consistent with a tighter binding constant. Furthermore, the more specificity in the design of a potential drug the more likely that the drug will not interact as well with other proteins. This will minimize potential side-effects due to unwanted interactions with other proteins.

Initially alternative compounds known to bind bacterial RNA polymerase, including rifampicin analogs, can be systematically modified by computer modeling programs until one or more promising potential analogs are identified. In addition systematic modification of selected analogs can then be systematically modified by computer modeling programs until one or more potential analogs are identified. Such analysis has been shown to be effective in the development of HIV protease inhibitors [Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)]. Alternatively a potential modulator could be obtained by initially screening a random peptide library produced by recombinant bacteriophage for example, [Scott and Smith, Science, 249:386-390 (1990); Cwirla et al., Proc. Natl. Acad. Sci., 87:6378-6382 (1990); Devlin et al., Science, 249:404-406 (1990)]. A peptide selected in this manner would then be systematically modified by computer modeling programs as described above, and then treated analogously to a structural analog as described below.

Once a potential modulator/inhibitor is identified it can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively the potential modulator may be synthesized de novo. The de novo synthesis of one or even a relatively small group of specific compounds is reasonable in the art of drug design. The potential modulator can be placed into a standard binding assay with RNA polymerase or an active fragment thereof, for example. The subunit fragments can be synthesized by either standard peptide synthesis described above, or generated through recombinant DNA technology or classical proteolysis. Alternatively the corresponding full-length proteins may be used in these assays.

For example, the β subunit can be attached to a solid support. Methods for placing the β subunit on the solid support are well known in the art and include such things as linking biotin to the β subunit and linking avidin to the solid support. The solid support can be washed to remove unreacted species. A solution of a labeled potential modulator (e.g., an inhibitor) can be contacted with the solid support. The solid support is washed again to remove the potential modulator not bound to the support. The amount of labeled potential modulator remaining with the solid support and thereby bound to the β subunit can be determined. Alternatively, or in addition, the dissociation constant between the labeled potential modulator and the β subunit, for example can be determined. Suitable labels for either the bacterial RNA polymerase subunit or the potential modulator are exemplified herein. In a particular embodiment, isothermal calorimetry can be used to determine the stability of the bacterial RNA polymerase in the absence and presence of the potential modulator.

In another embodiment, a Biacore machine can be used to determine the binding constant of the bacterial RNA polymerase to a DNA template in the presence and absence of the potential modulator. Alternatively, one or more of the bacterial RNA polymerase subunits can be immobilized on a sensor chip. The remaining subunits can then be contacted with (e.g., flowed over) the sensor chip to form the bacterial RNA polymerase.

In this case the dissociation constant for the bacterial RNA polymerase can be determined by monitoring changes in the refractive index with respect to time as buffer is passed over the chip [O'Shannessy et al. Anal. Biochem. 212:457-468 (1993); Schuster et al., Nature 365:343-347 (1993)]. Scatchard plots, for example, can be used in the analysis of the response functions using different concentrations of a particular subunit. Flowing a potential modulator at various concentrations over the bacterial RNA polymerase and monitoring the response function (e.g., the change in the refractive index with respect to time) allows the bacterial RNA polymerase dissociation constant to be determined in the presence of the potential modulator and thereby indicates whether the potential modulator is either an inhibitor, or an agonist of the bacterial RNA polymerase complex.

In another aspect of the present invention a potential modulator is assayed for its ability to inhibit the bacterial RNA polymerase. A modulator that inhibits the RNA polymerase can then be selected. In a particular embodiment, the effect of a potential modulator on the catalytic and/or transcriptional activity of bacterial RNA polymerase is determined. The potential modulator is then be added to a bacterial culture to ascertain its effect on bacterial proliferation. A potential modulator that inhibits bacterial proliferation can then be selected.

In a particular embodiment, the effect of the potential modulator on the catalytic and/or transcriptional activity of the bacterial RNA polymerase is determined (either independently, or subsequent to a binding assay as exemplified above). In one such embodiment, the rate of the DNA-dependent RNA transcription is determined. For such assays a labeled nucleotide could be used. This assay can be performed using a real-time assay e.g., with a fluorescent analog of a nucleotide. Alternatively, the determination can include the withdrawal of aliquots from the incubation mixture at defined intervals and subsequent placing of the aliquots on nitrocellulose paper or on gels. In a particular embodiment the potential modulator is selected when it is an inhibitor of the bacterial RNA polymerase.

One assay for RNA polymerase activity is a modification of the method of Burgess et al. [J. Biol. Chem., 244:6160 (1969)]
[See also http://www.worthington-biochem.com/manual/R/RNAP.html].

One unit incorporates one nanomole of UMP into acid insoluble products in 10 minutes at 37° C. under the assay conditions such as those listed below.

The suggested reagents are:
(a) 0.04 M Tris-HCl, pH 7.9, containing 0.01 M MgCl₂, 0.15 M KCl, and 0.5 mg/ml BSA;
(b) Nucleoside triphosphates (NTP): 0.15 mM each of ATP, CTP, GTP, UTP; spiked with ³H-UTP 75000-150000 cpms/0.1 ml;
(c) 0.15 mg/ml calf thymus DNA;
(d) 10% cold perchloric acid; and
(e) 1% cold perchloric acid.

0.1-0.5 units of RNA polymerase in 5 μl-10 μl is used as the starting enzyme concentration.

The procedure is to add 0.1 ml Tris-HCl, 0.1 ml NTP and 0.1 ml DNA to a test tube for each sample or blank. At zero time enzyme (or buffer for blank) is added to each test tube, and the contents are then mixed and incubated at 37° C. for 10 minutes. 1 ml of 10% perchloric acid is added to the tubes to stop the reaction. The acid insoluble products can be collected by vacuum filtration through MILLIPORE filter discs having a pore size of 0.45 u-10 u (or equivalent). The filters are then washed four times with 1% cold perchloric acid using 1 ml-3 ml for each wash. These filters are then placed in scintillation vials. 2 mls of methyl cellosolve are added to the scintillation vials to dissolve the filters. When the filters are completely dissolved (after about five minutes) 10 mls of scintillation fluid are added and the vials are counted in a scintillation counter.

For calculation of units of RNA polymerase/mg of protein the following equation can be used:

$$units \text{/} mg = \frac{{CPM}_{test} - {CPM}_{blank}}{{CPM}_{total} X mg {protein}_{in test}}$$

Alternative transcription assays can also be employed [see Example below, and Nudler et al., Science 265:793-796 (1994)]. One such assay comprises a core RNAP that can be incubated with a suitable σ subunit to form the holoenzyme. A potential modulator can then be added prior to, simultaneously with, subsequently to a promoter fragment (e.g., T7A1 as exemplified below). RNA synthesis is then initiated by the addition of a primer (e.g., a CpA primer) and the four nucleotide triphosphates (NTPs). The RNA synthesis in the presence and absence of the potential modulator is then quantified. In the Example below, a radioactive nucleotide was employed and the radioactive RNA products were analyzed on a 15% polyacrylamide sequencing gel. Alternatively, a fluorescent nucleotide analog can be used. Transcription reactions on a minimal scaffold system can be performed as shown in FIG. 6b below in the presence and the absence of the potential modulator [see also Korzheva et al., Science 289:619-625 (2000)].

When suitable potential modulators are identified, a supplemental crystal can be prepared which comprises the bacterial RNA polymerase and the potential modulator (see Example below). Preferably the crystal effectively diffracts X-rays for the determination of the atomic coordinates of the protein-ligand complex to a resolution of better than 5.0 Angstroms, more preferably equal to or better than 3.5 Angstroms. The three-dimensional structure of the supplemental crystal can be determined by Molecular Replacement Analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR (see above), CNS, (Crystallography and NMR System, a next level of XPLOR), and AMORE [J. Navaza, Acta Crystallographics ASO, 157-163 (1994)]. Once the position and orientation are known an electron density map can be calculated using the search model to provide X-ray phases. Thereafter, the electron density is inspected for structural differences and the search model is modified to conform to the new structure. Using this approach, it is also possible to use the claimed crystal of the Rif-RNAP complex to solve the three-dimensional structures of other bacterial core RNA polymerases bound to rifampicin (and/or other inhibitors) having pre-ascertained amino acid sequences. Other computer programs that can be used to solve the structures of the bacterial RNA polymerase from other organisms include: QUANTA, CHARMM; INSIGHT; SYBYL; MACROMODE; and ICM.

A candidate drug can be selected by performing rational drug design with the three-dimensional structure determined for the supplemental crystal, preferably in conjunction with computer modeling discussed above. The candidate drug (e.g., a potential modulator of bacterial RNA polymerase) can then be assayed as exemplified above, or in situ. A candidate drug can be identified as a drug, for example, if it inhibits bacterial proliferation.

A potential inhibitor (e.g., a candidate drug) would be expected to interfere with bacterial growth. Therefore, an assay that can measure bacterial growth may be used to identify a candidate drug.

Methods of testing a potential bactericidal agent (e.g., the candidate drug) in an animal model are well known in the art, and can include standard bactericidal assays. The potential modulators can be administered by a variety of ways including topically, orally, subcutaneously, or intraperitoneally depending on the proposed use. Generally, at least two groups of animals are used in the assay, with at least one group being a control group which is administered the administration vehicle without the potential modulator.

For all of the drug screening assays described herein further refinements to the structure of the drug will generally be necessary and can be made by the successive iterations of any and/or all of the steps provided by the particular drug screening assay.

### Labels

Suitable labels include enzymes, fluorophores e.g., fluorescein isothiocyanate (FITC), phycoerythrin (PE), Texas red (TR), rhodamine, free or chelated lanthanide series salts, especially Eu³⁺, to name a few fluorophores and including fluorescent GTP and GDP analogs such as mantGTP and mantGDP, chromophores, radioisotopes, chelating agents, dyes, colloidal gold, latex particles, ligands (e.g., biotin), and chemiluminescent agents. When a control marker is employed, the same or different labels may be used for the test and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized calorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017) Other direct labels include a radionucleotide, a luminescent moiety, or a fluorescent moiety including as a modified/fusion chimera of green fluorescent protein (as described in U.S. Pat. No. 5,625,048 filed Apr. 29, 1997, and WO 97/26333, published Jul. 24, 1997, the disclosures of each are hereby incorporated by reference herein in their entireties). In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419-439 (1980) and in U.S. Pat. No. 4,857,453.

Suitable enzymes include, but are not limited to, alkaline phosphatase and horseradish peroxidase. Other labels for use in the invention include magnetic beads or magnetic resonance imaging labels.

In another embodiment, a phosphorylation site can be created on an antibody of the invention for labeling with ³²P, e.g., as described in European Patent No. 0372707 (application No. 89311108.8) by Sidney Pestka, or U.S. Pat. No. 5,459,240, issued Oct. 17, 1995 to Foxwell et al.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

### Three-Dimensional Representation of the Structure of the Rif-RNAP complex

In addition, the present invention provides a computer that comprises a representation of the RNAP-RNAP binding partner complex (e.g., the Rif-RNAP complex) in computer memory that can be used to screen for compounds that will or are likely to inhibit RNAP. In a related embodiment, the computer can be used in the design of altered RNAPs that have either enhanced, or alternatively diminished RNA polymerase activity. Preferably, the computer comprises portions of and/or all of the information contained in Table 2. In a particular embodiment, the computer comprises: (i) a machine-readable data storage material encoded with machine-readable data, (ii) a working memory for storing instructions for processing the machine readable data, (iii) a central processing unit coupled to the working memory and the machine-readable data storage material for processing the machine-readable data into a three-dimensional representation, and (iv) a display coupled to the central processing unit for displaying the three-dimensional representation.

Thus the machine-readable data storage medium comprises a data storage material encoded with machine readable data which can comprise portions and/or all of the structural information contained in Table 2. One embodiment for manipulating and displaying the structural data provided by the present invention is schematically depicted in FIG. 7. As depicted, the System 1, includes a computer 2 comprising a central processing unit (“CPU”) 3, a working memory 4 which may be random-access memory or “core” memory, mass storage memory 5 (e.g., one or more disk or CD-ROM drives), a display terminal 6 (e.g., a cathode-ray tube), one or more keyboards 7, one or more input lines 10, and one or more output lines 20, all of which are interconnected by a conventional bidirectional system bus 30.

Input hardware 12, coupled to the computer 2 by input lines 10, may be implemented in a variety of ways. Machine-readable data may be inputted via the use of one or more modems 14 connected by a telephone line or dedicated data line 16. Alternatively or additionally, the input hardware 12 may comprise CD-ROM or disk drives 5. In conjunction with the display terminal 6, the keyboard 7 may also be used as an input device. Output hardware 22, coupled to computer 2 by output lines 20, may similarly be implemented by conventional devices. Output hardware 22 may include a display terminal 6 for displaying the three dimensional data. Output hardware might also include a printer 24, so that a hard copy output may be produced, or a disk drive 5, to store system output for later use, see also U.S. Pat. No. 5,978,740, Issued Nov. 2, 1999, the contents of which are hereby incorporated by reference in their entireties.

In operation, the CPU 3 (i) coordinates the use of the various input and output devices 12 and 22; (ii) coordinates data accesses from mass storage 5 and accesses to and from working memory 4; and (iii) determines the sequence of data processing steps. Any of a number of programs may be used to process the machine-readable data of this invention.

The present invention may be better understood by reference to the following non-limiting Example, which is provided as exemplary of the invention. The following example is presented in order to more fully illustrate the preferred embodiments of the invention. It should in no way be construed, however, as limiting the broad scope of the invention.

### EXAMPLE

### STRUCTURAL MECHANISM FOR RIFAMPICIN INHIBITION OF BACTERIAL RNA POLYMERASE

### Introduction

High-resolution structural studies of the Rif-RNAP complex should lead to insights into rifampicin binding, the mechanism of inhibition, and also the mechanism by which mutations lead to Rif^{R}. These structural studies will also shed light on the transcription mechanism itself, as well as provide the basis for the development of drugs that selectively inhibit bacterial RNAPs, but are less prone than rifampicin to lead bacterial mutations/substitutions of single amino acids that give rise to resistance.

Indeed, the recent determination of the crystal structure of core RNAP from Thermus aquaticus (Taq) [Zhang et al., Cell 98:811-824 (1999); U.S. Ser. No. 09/396,651, Filed Sep. 15, 1999, the contents of which are hereby incorporated by reference in their entireties] has opened the door to further studies of RNAP structure, function, and interactions with substrates, ligands, and inhibitors.

To further provide a more detailed framework to interpret the existing genetic, biochemical, and biophysical information, as well as to guide further studies aimed at understanding the transcription process and its regulation, the three-dimensional structure of a bacterial core RNAP complexed with rifampicin was determined by X-ray crystallography at 3.3 Å resolution as detailed below. The structure explains the effects of rifampicin on RNAP function. In combination with a model of the ternary transcription complex and biochemical experiments, the data indicate that the predominant effect of rifampicin on RNAP function is to directly block the path of the elongating RNA transcript at the 5′-end when the transcript becomes either 2 or 3 nucleotides in length.

### Methods

Purification and crystallization: Native Taq core RNAP was purified and crystallized as described previously [Zhang et al., Cell 98:811-824 (1999); U.S. Ser. No. 09/396,651, Filed Sep. 15, 1999, the contents of which are hereby incorporated by reference in their entireties]. Crystals were subsequently soaked in stabilization solution [2 M (NH₄)₂SO₄, 0.1 M Tris-HCl, pH 8.0, and 20 mM MgCl₂] with 0.1 mM rifampicin for at least 12 hours. The crystals were then prepared for cryo-crystallography by soaking in stabilization solution containing 50% (w/v) sucrose for 30 minutes before flash freezing in liquid nitrogen. Diffraction data was collected at the APS beamline SBC 19ID using 0.3° oscillations, and processed using DENZO and SCALEPACK [Otwinowski, Isomorphous Replacement and Anomalous Scattering (eds. Wolf, Evans and Leslie) Science and Engineering Research Council, Daresbury Laboratory, Daresbury, UK, (1991)].

In short, the preparative procedure for T. aquaticus core RNAP is similar to the preparation of E. coli core RNAP [Polyakov et al., Cell, 83:365-373 (1995)]. Briefly, approximately 200 g wet cell paste is thawed and lysed using a continuous-flow French press. After a low-speed spin, the soluble fraction is precipitated with 0.6% Polymin-P. RNAP is eluted from the Polymin-P pellet with TGED buffer (10 mM Tris —HCl, pH 8, 5% glycerol, 1 mM EDTA, 1 mM DTT) plus 1 M NaCl, then precipitated by adding 33%(g/v) ammonium sulfate. The pellet is resuspended and loaded onto a 50 ml column of heparin-SEPHAROSE FF (Pharmacia) equilibrated with TGED buffer plus 0.2 M NaCl. The RNAP is eluted from th e column with TGED buffer plus 0.6 M NaCl. The RNAP was again precipitated with ammonium sulfate, then resuspended and loaded on a SUPERDEX-200 gel filtration column equilibrated with TGED buffer plus 0.5 M NaCl. Fractions containing RNAP were pooled and loaded onto a MONO-Q (Pharmacia) ion-exchange column equilibrated with TGED buffer plus 0.1 M NaCl. The protein was eluted with a gradient from 0.1 to 0.5 M NaCl. The RNAP peak eluted at around 0.3 M NaCl. The RNAP was concentrated using a centrifugal filter, then loaded onto an SP SEPHAROSE (Pharmacia) column equilibrated in TGED buffer plus 0.1 M NaCl. After loading, the column was incubated at 4° C. for at least 10 hours, then pure RNAP was eluted with a 0.1 to 0.5 M NaCl gradient (core RNAP elutes at around 0.3 M NaCl). 200 g wet cell paste typically yielded 15 mg of core RNAP, which was more than 99% pure as judged from overloaded, Coomassie-stained SDS gels. This sample is ready for crystallization.

Crystals of T. aquaticus core RNAP were grown by vapor diffusion. 10 μl of T. aquaticus core RNAP (17 mg/ml) was mixed with the same volume of a solution containing 40-45% saturated (NH₄)₂SO₄, 0.1 M Tris-HCl, pH 8.0, and 20 mM MgCl₂, and incubated as a hanging drop over the same solution. Crystals grow in 2-3 weeks to typical dimensions of 0.15 mm×0.15 mm×0.4 mm at room temperature. For cryo-crystallography, the crystals are pre-soaked in stabilization solution (same as the crystallization solution except with 50% saturated ammonium sulfate). The crystals are then soaked in stabilization solution containing 50% (g/v) sucrose for about 30 minutes before flash freezing. The frozen crystals diffract to 5.0 Å from an in-house X-ray generator. Spots can sometimes be observed, in one direction, to 2.7 Å resolution at synchrotron beamlines. Diffraction data was processed using DENZO and SCALEPACK [Otwinowski, Isomorphous Replacement and Anomalous Scattering (eds. Wolf, Evans and Leslie) Science and Engineering Research Council, Daresbury Laboratory, Daresbury, UK, (1991)].

Selenomethionyl core RNAP was prepared and crystallized using the same procedures from T. aquaticus cells grown in minimal media (culture medium 162) [Degryse et al., Arch. Microbiol., 117:189-196 (1978)]. Cells were induced to incorporate selenomethionine by suppression of methionine biosynthesis [Doublie, Methods Enzymol., 276:523-530 (1997)].

Structure Determination: The native core RNAP structure [Zhang et al., Cell 98:811-824 (1999); U.S. Ser. No. 09/396,651, Filed Sep. 15, 1999, the contents of which are hereby incorporated by reference in their entireties] was used as a starting model for rigid body refinement and positional refinement against the observed amplitudes from the Rif-RNAP complex crystal (Fₒ^{Rif}:) using CNS [Adams et al., Proc. Natl. Acad. Sci. USA, 94:5018-5023 (1997)], yielding an initial R-factor of 0.354 (R_{free}=0.41, where the same set of reflections was set aside as was used for the R_{free} determination of the native structure) for data from 100-3.2 Å resolution. An initial Fourier difference map, calculated using |Fₒ^{Rif}−Fₒ^{nat}| amplitude coefficients and using phases calculated from the native core RNAP structure (φ^{nat}) clearly revealed density for the Rif molecule (FIG. 3a). Multiple rounds of manual rebuilding against (2|Fₒ|−|F_{c}|) maps using 0 [Jones et al., Acta Cryst, A 47:110-119 (1991)], and refinement using CNS [Adams et al., Proc. Natl. Acad. Sci. USA, 94:5018-5023 (1997)] resulted in the current model (Table 1). At later stages of the refinement, the Rif X-ray crystal structure [Brufani et al., J. Molec. Biol. 87:409-435 (1974)] was easily placed into the difference density. Included in the model is the recently determined sequence of the Taq ω subunit modeled earlier as a polyalanine chain [Zhang et al., Cell 98:811-824 (1999); U.S. Ser. No. 09/396,651, Filed Sep. 15, 1999, the contents of which are hereby incorporated by reference in their entireties]. Absent from the model is a 300 amino acid, non-conserved domain inserted between conserved regions A and B of the β′ subunit [Zhang et al., Cell 98:811-824 (1999); U.S. Ser. No. 09/396,651, Filed Sep. 15, 1999, the contents of which are hereby incorporated by reference in their entireties].

Assays: Taq cells were tested for sensitivity to rifampicin on solid media. Plates containing 3% bactoagar and ⅕ dilution of Luria broth were poured with and without 50 μg/ml of rifampicin. Cells from frozen stock were then streaked onto plates and incubated at 65° C. for 2 days and assessed for growth.

The transcription assay comparing rifampicin inhibition of E. coli and Taq RNAPs (FIG. 2a) was performed as previously described [Nudler et al., Science 265:793-796 (1994)]. Briefly, 0.1 pmol of purified Taq core RNAP [Zhang et al., Cell 98:811-824 (1999); U.S. Ser. No. 09/396,651, Filed Sep. 15, 1999, the contents of which are hereby incorporated by reference in their entireties] was incubated with Taq σ^{A} in 20 μl of transcription buffer (40 mM Tris-HCl, pH 7.9, 40 mM KCl, 5 mM MgCl₂) for 15 minutes at 37° C. to form holoenzyme. Rifampicin was added to the final concentrations indicated in FIG. 2a and incubated another 5 minutes at 37° C., followed by the addition of 0.15 pmol of T7A1 promoter fragment and incubation for 5 minutes at 37° C. RNA synthesis was initiated by the addition of CpA primer (100 μM), NTPs (25 μM each), and α-[³²P]UTP (0.3 μM), and the reaction was stopped after incubation for 10 minutes at 37° C. The assay for E. coli RNAP holoenzyme was the same except the CpA primer was added to a concentration of 10 μM. Radioactive RNA products were analyzed on a 15% polyacrylamide sequencing gel.

Assays for extension of the Rif-nucleotide compounds (FIG. 2c-2d) were carried out as described [Mustaev et al., Proc. Nat. Acad. Sci. USA 91:12036-12040 (1994)] with minor modifications. After binary complex formation, transcription reactions were started by the addition 10 μM Rif-(CH2)ₙ-A compound, with the ‘n’ indicated in FIG. 2c-2d, and α-[³²P]UTP (0.3 μM). The reactions were incubated for 2 minutes at room temperature for E. coli RNAP and 3 minutes at 55° C. for Taq. Under these conditions, the reaction was not complete, and the yield of the Rif-(CH2)ₙ-ApU depended on the linker length. Radioactive RNA products were analyzed on a 23% polyacrylamide sequencing gel.

Transcription reactions on the minimal scaffold system shown (FIG. 6b) were performed as described [Korzheva et al., Science 289:619-625 (2000)] with minor modifications. The RNA and DNA components of the scaffold (100 pmol of each) were mixed in 100 μl of transcription buffer at 45° C. and the mixture was allowed to cool to room temperature over 30 minutes. RNAP/scaffold complexes were formed by incubation of the annealed scaffold (10 pmol) with a molar equivalent of core RNAP (either E. coli or Taq) which was preincubated with rifampicin (100 μM for E coli, 200 μM for Taq) for 10 minutes to form the RNAP/scaffold complex. Extension of the RNA oligonucleotide was assayed by the addition of α-[³²P]CTP (0.3 μM) and a 5 minute incubation at room temperature. In FIG. 6b, lanes 1-5 and 16-20, RNAP was preincubated with rifampicin (100 μM for E. coli RNAP, 200 μM for Taq) for 10 minutes. In lanes 6-10 and 21-25, the RNAP/scaffold complexes formed in the absence of rifampicin were incubated with rifampicin (concentrations as above) for 10 minutes. Finally, in lanes 11-15 and 26-30, the RNAP or RNAP/scaffold complex was not exposed to rifampicin. Radioactive RNA products were analyzed on a 23% polyacrylamide sequencing gel.

### Results

Rifampicin Inhibition of Taq RNAP: From a biochemical perspective, the interaction of rifampicin (Rif) with RNAP has been extensively characterized using E. coli RNAP, which served as a prototype for bacterial pathogens [Drancourt and Raoult, Antimicr. Agents Chemother. 43:2400-2403 (1999); Heep et al., Antimicr. Agents Chemother. 44:1075-1077 (1999); Honore et al., Molec. Microbiol. 7:207-214 (1993); Morse et al., J. Clin. Microbiol, 37:2913-2929 (1999); Nolte, J. Antimicrob. Chemother 39:747-755(1997); Padayachee and Klugman, Antimicr. Agents Chemother. 43:2361-2365 (1999); Ramaswamy and Musser, Tubercle and Lung Disease 79:3-29 (1998); Wichelhaus et al., Antimicr. Agents Chemother. 43:2813-2816(1999)]. The inhibition of Taq RNAP by rifampicin was therefore investigated to assess this system as a structural model for Rif-RNAP interactions. Sequence comparisons in the four distinct regions of rpoB which harbor Rif^{R} mutations indicate a very high level of conservation among prokaryotes. Between E. coli, Taq, and M. tuberculosis, the sequences are 91% identical over 60 residues (93% conserved), explaining the broad spectrum of rifampicin activity. Nevertheless, among the 23 positions with single amino-acid substitutions that give rise to Rif^{R} in either E. coli or M. tuberculosis, 5 of these positions (Taq p 387, 395, 398, 453, and 566; the Taq numbering is used throughout this application unless otherwise specified) are substituted in Taq (FIG. 1). In contrast, there is a relatively low level of conservation between prokaryotes and eukaryotes within these regions (FIG. 1), explaining the lack of rifampicin activity against eukaryotic RNAPs and eukaryotic cells.

A plate assay (see Methods above) was used to show that Taq cells were unable to grow on media supplemented with 50 μg/ml rifampicin. For in vitro studies, Taq RNAP holoenzyme was reconstituted using Taq core RNAP purified from Taq cells [Zhang et al., Cell 98:811-824 (1999); U.S. Ser. No. 09/396,651, Filed Sep. 15, 1999, the contents of which are hereby incorporated by reference in their entireties] and recombinant Taq σ^{A} (overexpressed and purified from E. coli). The enzyme initiated, elongated, and terminated transcripts efficiently from a template containing the T7A1 promoter and the tR2 intrinsic terminator (FIG. 2a) [Nudler et al., J. Molec. Biol. 288:1-12(1994)] at 37° C. using the dinucleotide CpA as the initating primer. The major RNA products, a trimeric abortive transcript (CpApU), a 105 nucleotide terminated transcript (Term), and a 127 nucleotide runoff transcript (Run off), were the same as those produced by E. coli RNAP (FIG. 2a, lanes 1 and 8). Since E. coli σ⁷⁰ is totally inactive when combined with Taq core RNAP in this assay, the possibility of trace contamination with E. coli σ⁷⁰ does not affect the conclusions from this assay for Taq RNAP. Quantitatively, the two RNAPs responded very differently to rifampicin, the Ki (estimated from the rifampicin concentration where the production of long transcripts was inhibited by 50%) for E. coli RNAP was about 0.1 μM, while for Taq RNAP it was about 10 μM, a 100-fold difference in sensitivity. Qualitatively, however, both RNAPs responded the same way, with an increase in the production of the trimeric product and a concurrent precipitous drop in the production of the long transcripts (FIG. 2a).

Mustaev et al., [Proc. Nat. Acad. Sci. USA 91:12036-12040 (1994)] used chimeric Rif-nucleotide compounds to measure the distance between the initiating nucleotide binding site (the i-site) and the Rif binding site. By varying the linker between the Rif and the nucleotide and testing for maximal transcription initiation activity, the optimal length was found that allowed binding of each moiety in its respective site. This experiment was used to compare the disposition of the Rif and i-sites in E. coli and Taq RNAP. In both cases, optimal initiation activity was observed when the linker comprised five —(CH2)— groups (FIGS. 2c-2d). Thus, in spite of the fact that Taq RNAP requires a 100-fold higher concentration of rifampicin for inhibition, Taq. RNAP binds rifampicin and is inhibited through the same biochemical mechanism as E. coli RNAP, and the disposition of the Rif-site with respect to the universally conserved active site is identical. Therefore, Taq RNAP can serve as a model for rifampicin interactions with other RNAPs.

Rif-RNAP Structure Determination and Refinement: Tetragonal crystals of Taq core RNAP [Zhang et al., Cell 98:811-824 (1999); U.S. Ser. No. 09/396,651, Filed Sep. 15, 1999, the contents of which are hereby incorporated by reference in their entireties] were incubated overnight in stabilization buffer with 0.1 mM rifampicin, followed by a 30 minute soak in cryo-solution (without rifampicin) before flash freezing. During this procedure, the crystals took on a deep orange color, confirming the binding of rifampicin. The same results were obtained with co-crystals grown in the presence of 0.1 mM rifampicin, suggesting that rifampicin binding causes few if any conformational changes in the RNAP.

The Taq core RNAP:Rif crystals were isomorphous with the native Taq core RNAP crystals [Zhang et al., Cell 98:811-824 (1999); U.S. Ser. No. 09/396,651, Filed Sep. 15, 1999, the contents of which are hereby incorporated by reference in their entireties]. Strong electron density was observed in difference Fourier maps for the rifampicin (FIG. 3a), which occupies a shallow pocket between β structural domains 3 and 4 (FIG. 3b) that is surrounded by the known Rif^{R} mutations (FIG. 1) [Zhang et al., Cell 98:811-824 (1999); U.S. Ser. No. 09/396,651, Filed Sep. 15, 1999, the contents of which are hereby incorporated by reference in their entireties]. The electron difference density also indicated shifts and/or ordering of several p residues interacting directly with rifampicin, including Q390, L391, Q393, D396, H406, R409, and L413 (FIG. 4). Only very small shifts in localized regions of the protein backbone were indicated.

The rifampicin X-ray crystal structure [Brufani et al., J. Molec. Biol. 87:409-435 (1974)] was easily placed into the difference density. Subsequent refinements resulted in only small shifts of the ansa chain (FIG. 3c) to better fit the density. Multiple rounds of manual rebuilding against (2|Fₒ|−|F_{c}|) maps and refinement resulted in the current model (see Methods above and Table 1).

TABLE 1
CRYSTALLOGRAPHIC DATA AND STRUCXTURAL MODEL
DIFFRACTION DATA

| Parameter | Total | Outer Shell |
|---|---|---|
| Resolution range (Å) | 30-3.3 | 3.42-3.3 |
| Rmerge1 (%) | 7.7 | 34.4 |
| Completeness (%) | 86.1 | 71.7 |
| I/σI | 10.7 | 1.7 |
| No. of reflection | 75,240 | 6,173 |
| No. of unique obs. | 214,453 | 11,549 |

STRUCTURAL MODEL
Number of Residues

| Protein | | | | |
|---|---|---|---|---|
| Subunit² | Mr (kDa) | sequence | model | regions modeled |
| β′ | 170.7 | 1,525 | 1,139 | 3-31, 69-115 (poly-Ala), |
| | | | | 452-523, 536-1241, |
| | | | | 1250-1410, 1414-1497 |
| β | 124.4 | 1,119 | 1,114 | 2-1115 |
| αI | 34.9 | 313 | 223 | 6-228 |
| αII | 34.9 | 313 | 229 | 3-231 |
| ω | 11.6 | 99 | 98 | 1-98 |
| Total | 376.5 | 3,369 | 2,803 | |

REFINEMENT

| | |
|---|---|
| R_{cryst} (%) | 28.1 |
| R_{free} (%) | 35.9 |

¹Rmerge = Σ|Ij − <I>|/ΣIj
²Also included in the model was one Mg²⁺ and one Zn²⁺ ion [Zhang et al., Cell 98:811-824 (1999); U.S. Ser. No. 09/396,651, Filed September 15, 19999] and one Rif molecule [Brufani et al., J. Molec. Biol. 87:409-435 (1974)].

Overall Structure: Consistent with the fact that all mapped Rif^{R} mutants occur in rpoB (FIG. 1), rifampicin makes contacts only with the RNAP β subunit in a close complementary fit to its binding pocket deep within the main DNA/RNA channel. Clearly, rifampicin does not bind directly at the RNAP active site (FIG. 3b). The closest approach of rifampicin to the active site, defined as the distance between the active site Mg²⁺ and C38 of rifampicin (see FIG. 3c), is 12.1 Å.

Detailed Interactions: A large number of rifampicin derivatives have been investigated for antimicrobial activity. In general, modification of the ansa bridge, or modifications that alter the conformation of the ansa bridge, reduce activity. Other structural features of the antibiotic that are particularly critical for activity include the napthol ring with oxygen atoms (O1 and O2) at C1 and C8, and unsubstituted hydroxyls (O10 and O9) at C21 and C23 (see FIG. 3c) [Arora, Acta Crystall. B37:152-157 (1981); Arora, Molecular Pharmacology 23: 133-140 (1983); Arora, J. Med. Chem. 28:1099-1102 (1985); Arora and Main, J. Antibiot. 37:178-181 (1984); Brufani et al., J. Molec. Biol. 87:409-435 (1974); Lancini and Zanichelli, In Structure-activity Relationship in Semisynthetic Antibiotics, D. Perlaman, ed. (Academic Press), pp. 531-600 (1977); Sensi et al., Rev. Infect. Dis., 5 Supp.3:402-406 (1983)]. Most rifampicin modifications that retain activity involve substitutions at C3 of the napthol ring, which have only modulatory effects on in vitro activity.

These results can be explained by the structural details of the Rif-RNAP complex (FIGS. 4a-4b and 5a-5b). A cluster of hydrophobic residues (L391; L413, G414, I452) line one wall of the Rif binding pocket and make van-der-Waals contact with the napthol ring and the methyl group at C7. One end of the binding pocket (the bottom in FIGS. 4a-4b) is formed by Q390. The alkyl chain of Q390 makes van-der-Waals contact with Rif C28 and C29, while the polar head group may interact with 05. Protein groups are positioned to make hydrogen bonds with each of the four critical hydroxyls of rifampicin: R409 with O1, Q393 and S411 with O2, and D396 and H406 with O10. O9 and O10 are also in position to interact with the backbone amide and carboxyl of F394, respectively. O8 of rifampicin is also positioned to make a potential hydrogen bond with the backbone amide of F394.

D396 contributes to the binding interface in several ways. In addition to forming a potential hydrogen bond with O10 of rifampicin, it forms the top end of the binding pocket (in FIGS. 4a-4b) by making van-der-Waals contact with C18-C21, and C31. Moreover, the negative charge of D396 may be important for neutralizing the positive charges of two nearby side chains, R405 and R409 (FIGS. 4a-4b), each about 6 Å away. The charge neutralization might be important for the binding of the relatively apolar of rifampicin. Most Rif^{R} mutants at amino acid residue396 substitute a large, bulky group that would likely interfere with rifampicin binding and would not have the correct geometry for hydrogen bonding O10 (Y), or else substitute an apolar group (V, G, or A) with no hydrogen bonding ability. One of these mutants, D396V (amino acid position 516 in E. coli), was among the original, strong Rif^{R} mutants mapped by Ovchinnikov et al. [Molec. Gen. Genet. 190:344-348 (1983)], pointing to the importance of this residue in forming the rifampicin binding interface. Another mutant identified in E. coli, however (D396N), is isosteric with aspartic acid and would likely maintain the hydrogen bond with O10. Nevertheless, this substitution yields weak Rif^{R} [Lisitsyn et al., Bioorg Khim 10:127-128 (1984)], which is likely caused by the loss of the negative charge at this position.

Rifampicin has a partial +−charge, localized at N4 (FIG. 3c). A negatively-charged residue, E445, is situated nearby and may contribute to the rifampicin binding site by neutralizing this charge. This is not likely to be a strong effect, as many rifampicin derivatives with equal or stronger activity than rifampicin do not have this partial charge. E445 is the only residue close enough to rifampicin to be involved in potentially direct interactions (FIGS. 4a-4b) for which a Rif^{R} mutant has not been reported. However, this residue is universally conserved as either glutamic acid or aspartic acid in a segment of β region D that is invariantly present in prokaryotes, chloroplast, archaebacteria, and eukaryotes [Allison et al., Cell 42:599-610 (1985); Sweetser et al., Proc. Natl. Acad. Sci. USA 84:1192-1196 (1987)], pointing to its importance for the basic function of RNAP.

Thus, of the 12 residues that are close enough to rifampicin to make direct interactions (including backbone interactions with F394; FIGS. 4a-4b), 11 mutate to a Rif^{R} phenotype. The twelfth position, E445, is highly conserved so that its substitution would likely be lethal and consequently not be detectable as a Rif^{R} mutation.

Twelve additional positions have been identified at which substitution gives rise to Rif^{R} (FIG. 1). These residues surround the Rif binding pocket but do not make direct interactions with the antibiotic (FIGS. 5a-5b). In every case, the Rif^{R} mutations involve replacement by a different sized amino acid side-chain (almost always substituting a small residue with a more bulky one), or else involve adding or removing a proline residue. These substitutions would likely affect the folding or packing of the protein in the local vicinity of the substituted residue, causing distortions of the Rif binding pocket.

Mechanism of RNAP Inhibition by Rif. The effects of rifampicin on RNAP in each stage of the transcription cycle have been probed using detailed kinetic analyses. Rifampicin has essentially no effect on specific promoter binding and open complex formation [Hinkle et al., J. Molec. Biol., 70, 209-220 (1972); McClure and Cech, J. Biol. Chem. 253:8949-8956 (1978)]. A small increase (about 2-fold) in the apparent Km for initiating substrate binding in the enzyme's i-site (the 5′-nucleotide) was observed, but the binding of the incoming nucleotide substrate in the i+1 site (the 3′-nucleotide), and the formation of the first phosphodiester bond were largely unaffected [McClure and Cech, J. Biol. Chem. 253:8949-8956 (1978)]. The dominant effect of rifampicin binding on RNAP activity was a total blockage of synthesis of the second (when transcription was initiated with a nucleoside triphosphate) or third (when transcription was initiated with a nucleoside di- or monophosphate) phosphodiester bond [McClure and Cech, J. Biol. Chem. 253:8949-8956 (1978)]. Since synthesis of the first and second phosphodiester bond can occur in the presence of rifampicin, the antibiotic does not interfere with substrate binding, catalytic activity, or the intrinsic translocation mechanism of the RNAP. After RNAP has synthesized a long transcript and entered the elongation phase, it becomes totally resistant to rifampicin. These properties led to the proposal that rifampicin inhibits RNAP through a simple steric block of the path of the elongating RNA at the 5′-end [McClure and Cech, J. Biol. Chem. 253:8949-8956 (1978)]. Whether rifampicin directly blocked the path of the RNA, or if blockage was an indirect effect due to a conformational change in the RNAP induced by rifampicin binding, could not be distinguished. It has alternatively been proposed that rifampicin exerts its effect allosterically by decreasing the affinity of the RNAP for short RNA transcripts [Schulz and Zillig, Nucl. Acids Res. 9:6889-6906 (1981)].

The Rif-RNAP crystal structure explains the results described above and strongly supports the simple steric block mechanism, see, atomic coordinates included in Table 2 [McClure and Cech, J. Biol. Chem. 253:8949-8956 (1978)]. Rifampicin directly abuts the base of a loop that comprises the C-terminal part of the β conserved region D (amino acid residues 443-451, shaded red in FIGS. 5a-5b), and a cluster of Rif^{R} mutants, Rif cluster I (FIG. 1), flanks this region. Modeling suggests that this loop, which contains several nearly universally conserved residues, participates in forming the binding site for the base-pair at +1 in the transcription complex [Korzheva et al., Science 289:619-625 (2000)], so effects of rifampicin on the Km for the initiating substrate are not surprising. However, rifampicin does not directly contact the end of this loop. In addition, conformational changes of the protein in this region are not indicated from the structural data, consistent with the observation that the effect of rifampicin on this region is small.

The principal effect of rifampicin is seen in the context of a model of the transcriptionally active ternary complex [Korzheva et al., Science 289:619-625 (2000)] containing RNAP, DNA template, and RNA transcript (FIG. 6a). In FIG. 6, only the RNAP active site Mg²⁺ and the 9-basepair RNA/DNA hybrid (from +1 to −7) from the ternary complex model are shown. The rest of the RNAP and nucleic acids are omitted for clarity. Also shown is the atomic model of rifampicin as it would be positioned in its binding site on the β subunit.

It can be seen that the two substrate nucleotides, at +1 (green) and −1, are not directly affected by the presence of rifampicin so that RNAP can bind and catalyze the formation of a phosphodiester bond between the two substrates in the presence of the antibiotic. With a transcript length of 3 nucleotides (nt), however, the 5′-phosphates of the 5′-nucleotide (at −2) sterically clash with rifampicin, and the nucleotides further upstream (−3 to −5) severely clash with rifampicin. At the same time, rifampicin does not interfere with the DNA (grey). Thus, the structure, in combination with the ternary complex model, explains the biochemical data on the mechanism of rifampicin inhibition, provides strong support for the proposal that rifampicin sterically blocks the path of the elongating RNA transcript at the 5′-end, and indicates that the blockage is a direct consequence of rifampicin binding in its site. The model further suggests why transcripts initiated with nucleoside triphosphates are blocked after the first phosphodiester bond, while transcripts initiated with nucleoside di- or monophosphates are blocked after the second phosphodiester bond. In the model, the nucleoside monophosphate in the transcript at the −2 position clashes only slightly with rifampicin, while the presence of a 5′-triphosphate at the −2 position would extend into rifampicin.

Core RNAP can bind a pre-formed ‘minimal nucleic acid scaffold’ of RNA/DNA oligonucleotides (FIG. 6b, top) to yield functional ternary elongation complexes [Korzheva et al., Science 289:619-625 (2000)]. Order of addition experiments were performed using this system in order to assess whether rifampicin and RNA binding were competetive (FIG. 6b). The DNA component of the scaffold was annealed with varying lengths of RNA transcript, and the effect of rifampicin on the sequence-dependent extension of RNA by one nucleotide (radioactively-labeled CTP) added before or after the oligonucleotides was assayed at room temperature. In the case of E. coli core RNAP in the absence of rifampicin the RNA transcript was extended with nearly equal efficiency regardless of its length within a range of 3-7 nucleotides (FIG. 6b, lanes 11-15). When rifampicin was added prior to the nucleotide scaffold, the RNAP was unable to extend any of the RNA oligos, regardless of length (lanes 1-5), indicating that rifampicin occupied its site and blocked the extension and/or binding of all of the transcripts. When the scaffold was added prior to rifampicin addition, rifampicin was able to occupy its site and block the extension of the 3-nucleotide transcript (lane 6), but had no effect on the extension of the longer transcripts (lanes 7-10), presumably because rifampicin could not access its binding site due to the presence of the longer RNA transcripts (FIG. 6a). This result is consistent with the early data that rifampicin inhibits the RNA extension from 2 to 3 nucleotides if the 5′-nucleoside is tri-phosphorylated, but inhibits extension from 3 to 4 nucleotides if the 5′-nucleoside is mono- or di-phosphorylated [McClure and Cech, J. Biol. Chem. 253:8949-8956 (1978)] since the synthetic RNA oligos lack 5′-phosphates.

Similar experiments were performed with Taq core RNAP (FIG. 6b, lanes 16-30). In the absence of rifampicin, the efficiency of transcript extension was strongly dependent on the transcript length (lanes 26-30). Extension of the shortest transcripts was barely detectable, suggesting that, unlike E. coli RNAP, Taq, core RNAP does not bind and stabilize the short, intrinsically unstable RNA/DNA hybrids. In the presence of rifampicin, a generalized inhibition of transcript extension was observed regardless of the order of addition or of the transcript length (lanes 16-25). These results can be explained by the low binding affinity of Taq core RNAP for both rifampicin and for short RNA transcripts compared with E. coli core RNAP. The low affinities imply fast off-rates, which would allow equilibrium to be established between the rifampicin and scaffold binding during the time of the assay.

### Discussion

The 3.3 Å X-ray crystal structure of Taq core RNAP complexed with rifampicin is disclosed herein. Though Taq RNAP is less sensitive to rifampicin than E. coli rifampicin, at sufficiently high concentrations the antibiotic binds and inhibits the enzyme. Significantly, however, the inhibition of Taq RNAP by rifampicin occurs through the same biochemical mechanism as E. coli RNAP, and the disposition of the Rif-site with respect to the active site is identical to E. coli RNAP as well as with other prokaryotic RNAPs (FIGS. 2a-2d). Therefore, the structural information provided herein is relevant for all bacteria RNAPs.

The relative insensitivity of Taq RNAP to rifampicin is likely due to amino acid substitutions in Taq RNAP compared with other, more Rif-sensitive RNAPs. The 12 residues close enough to interact directly with the rifampicin are identical between E. coli, Taq, and M. tuberculosis (marked yellow in FIG. 1). Among the 11 secondary positions that do not directly interact with rifampicin but likely affect rifampicin binding indirectly, 5 are substituted in Taq RNAP (amino acid residues 387, 395, 398, 453, and 566; FIG. 1). Three of these positions, 387, 398, and 453, contain amino acids that are not dramatically different in overall size from their E. coli and M. tuberculosis counterparts and one would predict that these residues are not the origin of the Taq RNAP insensitivity to rifampicin. Position 566 is highly conserved among all RNAPs as either a lysine or an arginine (the homologous position is an arginine in both E. coli and M. tuberculosis) but is a threonine in Taq RNAP. This substitution is unlikely to be the main determinant of the Taq RNAP Rif insensitivity, however, since mutating Taq Thr566 to an arginine has little effect on the Rif^{R} of the enzyme when assayed at 45° C. This leaves position 395, which is highly conserved as a hydrophobic residue among all RNAPs. In E. coli and M. tuberculosis this position is a methionine, but in Taq it is a lysine. Taq Lys395 appears to participate in buried salt-bridges with Asp124 and Asp133 that may contribute to the thermostability of the protein. This non-conservative substitution (lysine for methionine) could affect the local path of the polypeptide backbone, and is immediately adjacent to Phe394, the backbone amide and carboxyl of which appear to be involved in important interactions with the rifampicin (FIGS. 4a-4b).

All but one of the residues that are close enough to rifampicin to participate in direct interactions are known to mutate to strong Rif^{R} (FIGS. 4a-4b). However, additional residues could be important for the formation of the Rif binding pocket but not revealed as Rif^{R} mutants if they are necessary for basic RNAP function. As mentioned above, the four regions of the β subunit that harbor Rif^{R} mutants are highly conserved among prokaryotes (FIG. 1), but the much weaker homology with archaebacterial and eukaryotic RNAPs, combined with the fact that so many Rif^{R} mutations have been discovered, indicate that these regions are not critical to RNAP function in vivo. Nevertheless, some Rif^{R} mutations do have profound functional effects [Jin and Gross, J. Biol. Chem. 266:14478-14485 (1991); Landick et al., Genes Develop. 4:1623-1636 (1990)], and E. coli strains with Rif^{R} RNAP have been shown to be at a competetive disadvantage to wild type E. coli in the absence of rifampicin [Jin and Gross, J. Bact. 171:5229-5231 (1989)].

The clinical success of rifampicin proves that the bacterial RNAP is an excellent target for antimicrobials. The structure and available genetic and biochemical data suggest that the design of modified versions of rifampicin to overcome the effects of Rif^{R} mutations may lead to incremental improvements, though may not lead to a “wonder” drug because of the apparently small functional penalties of mutating this region of the RNAP, and the variety of amino acid positions and mutations that result in Rif^{R} (FIG. 1). In contrast, however, the findings from clinical isolates of Rif^{R} M. tuberculosis are rather encouraging. Thus, although the Rif^{R} mutations are spread over 15 positions of rpoB, 77% of all the mutations isolated involved substitutions at one of only two positions, corresponding to Taq amino acid residues 406 and 411. If a third amino acid residue is included, i.e., (Taq 396) a combined 86% of all the reported mutants are accounted for.

One important conclusion from the present disclosure emerges regarding the inhibitory mechanism of rifampicin, i.e., it is a simple steric block of transcription elongation. Thus, the powerful effects of rifampicin do not stem from the details of its chemical structure, and do not involve interference with the catalytic activity of the RNAP, e.g., by mimicking substrates or a transition state of the polymerization reaction. Indeed, such an inhibitor would likely act on features that are highly conserved between prokaryotes and eukaryotes, rendering the inhibitor useless as an antimicrobial agent. Rather, the effects of rifampicin depend only on its ability to bind tightly to a relatively non-conserved part of the structure, disrupting a critical RNAP function by virtue of its presence. Decades of functional studies [Chamberlin, Harvey Lectures 88:1-21 (1993); Korzheva et al., Cold Spring Harbor Symposia on Quantitative Biology 63:337-345 (1998); Mustaev et al., Proc. Nat. Acad. Sci. USA 91:12036-12040 (1994); and Nudler, J. Molec. Biol. 288:1-12 (1999)], and more recent structural evidence [Cramer et al., Science 288:640-649 (2000); Korzheva et al., Science 289:619-625 (2000); Mooney and Landick, Cell 98:687-690(1999); Zhang et al., Cell 98:811-824 (1999); U.S. Ser. No. 09/396,651, Filed Sep. 15, 1999, the contents of which are hereby incorporated by reference in their entireties] indicate that cellular RNAPs operate as complex molecular machines, with extensive interactions with the template DNA, product RNA [Korzheva et al., Science 289:619-625 (2000)], and other regulatory molecules. Thus, many additional distinct sites exist where the tight binding of a small molecule (i.e., a novel antibiotic) would disrupt critical features of the functional mechanism of bacterial RNAPs. Such distinct sites can be readily identified through the structural information provided by the present invention.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

1. A crystal of rifampicin bound to a core RNA polymerase (Rif-RNAP) that effectively diffracts X-rays for the determination of the atomic coordinates to a resolution of better than 3.5 Angstroms.

2. The crystal of claim 1, wherein the core RNA polymerase is a bacterial core RNA polymerase.

3. The crystal of claim 2, wherein the bacterial core RNA polymerase is a thermophilic bacterial core RNA polymerase.

4. The crystal of claim 3, wherein the thermophilic bacterial core RNA polymerase is a Thermus aquaticus bacterial core RNA polymerase.

5. The crystal of claim 1, wherein the core RNA polymerase comprises a β′ subunit, a β subunit, and a pair of α subunits.

6. The crystal of claim 5, further comprising an ω subunit.

7. The crystal of claim 1 that effectively diffracts X-rays for the determination of the atomic coordinates of the core RNA polymerase to a resolution of 3.3 Angstroms or better.

8. The crystal of claim 7 having space group of P4₁2₁2 and a unit cell of dimensions of a=b=201 and c=294 Å.

9. A method of identifying an agent for use as an inhibitor of bacterial RNA polymerase comprising:
(a) obtaining a set of atomic coordinates defining the three-dimensional structure of rifampicin bound to the core RNA polymerase (Rif-RNAP); wherein said core RNAP consists essentially of the β′, β, α and ω subunits of RNAP from T. aquaticus and using a crystal having the space group of P4₁2₁2 and unit cell dimensions of a=b=201 and c=294 Å;
(b) selecting a potential agent by performing rational drug design with the atomic coordinates obtained in step (a), wherein said selecting is performed in conjunction with computer modeling;
(c) contacting the potential agent with a bacterial RNA polymerase; and
(d) measuring the activity of the bacterial RNA polymerase; wherein a potential agent is identified as an agent that inhibits bacterial RNA polymerase when there is a decrease in the activity of the bacterial RNA polymerase in the presence of the agent relative to in its absence.

10. The method of claim 9, further comprising:
(e) preparing a supplemental crystal containing the core RNA polymerase formed in the presence of the potential agent, wherein the crystal effectively diffracts X-rays for the determination of the atomic coordinates to a resolution of better than 5.0 Angstroms;
(f) determining the three-dimensional coordinates of the supplemental crystal with molecular replacement analysis; and
(g) selecting a second generation agent by performing rational drug design with the three-dimensional coordinates determined for the supplemental crystal, wherein said selecting is performed in conjunction with computer modeling.

11. The method of claim 10, further comprising:
(h) contacting the second generation agent with a eukaryotic RNA polymerase; and
(i) measuring the activity of the eukaryotic RNA polymerase; wherein an agent is identified as an agent for use as an inhibitor of bacterial RNA polymerase when there is no change in the activity of the eukaryotic RNA polymerase in the presence of the agent relative to in its absence; and wherein the agent identified inhibits bacterial but not eukaryotic RNA polymerase.

12. A method of identifying an agent that inhibits bacterial growth comprising:
(a) obtaining a set of atomic coordinates defining the three-dimensional structure of rifampicin bound to core RNA polymerase (Rif-RNAP); wherein the core RNA polymerase consists essentially of the β′, β, α and ω subunits of RNAP from T. aquaticus and using a crystal having the space group of P4₁2₁2 and unit cell dimensions of a=b=201 and c=294 Å;
(b) selecting a potential agent by performing rational drug design with the atomic coordinates obtained in step (a), wherein said selecting is performed in conjunction with computer modeling;
(c) contacting the potential agent with a bacterial culture; and
(d) measuring the growth of the bacterial culture under conditions in which the bacterial culture grows in the absence of the agent; wherein a potential agent is identified as an agent that inhibits bacterial growth when there is a decrease in the growth of the bacterial culture in the presence of the agent relative to in its absence.

13. The method of claim 12, further comprising:
(e) preparing a supplemental crystal containing the core RNA polymerase formed in the presence of the potential agent, wherein the crystal effectively diffracts X-rays for the determination of the atomic coordinates to a resolution of better than 5.0 Angstroms;
(f) determining the three-dimensional coordinates of the supplemental crystal with molecular replacement analysis; and
(g) selecting a second generation agent by performing rational drug design with the three-dimensional coordinates determined for the supplemental crystal, wherein said selecting is performed in conjunction with computer modeling.

14. The method of claim 13, further comprising:
(h) contacting the second generation agent with a eukaryotic cell; and
(i) measuring the amount of proliferation of the eukaryotic cell under conditions in which the eukaryotic cell proliferates in the absence of the agent; wherein an agent is identified as an agent for inhibiting bacterial growth when there is no change in the proliferation of the eukaryotic cell in the presence of the agent relative to in its absence; and wherein the agent identified inhibits bacterial growth but not eukaryotic proliferation.

15. A method of identifying an agent for use as an inhibitor of bacterial RNA polymerase comprising:
(a) selecting a potential agent by performing rational drug design with the set of atomic coordinates in Table 2, wherein said selecting is performed in conjunction with computer modeling;
(b) contacting the potential agent with a bacterial RNA polymerase; and
(c) measuring the activity of the bacterial RNA polymerase; wherein a potential agent is identified as an agent that inhibits bacterial RNA polymerase when there is a decrease in the activity of the bacterial RNA polymerase in the presence of the agent relative to in its absence.

16. The method of claim 15, further comprising:
(d) preparing a crystal containing a bacterial RNA polymerase formed in the presence of the potential agent, wherein the crystal effectively diffracts X-rays for the determination of the atomic coordinates to a resolution of better than 5.0 Angstroms;
(e) determining the three-dimensional coordinates of the crystal with molecular replacement analysis; and
(f) selecting a second generation agent by performing rational drug design with the three-dimensional coordinates determined for the crystal, wherein said selecting is performed in conjunction with computer modeling.

17. The method of claim 16, further comprising:
(g) contacting the second generation agent with a eukaryotic RNA polymerase; and
(h) measuring the activity of the eukaryotic RNA polymerase; wherein an agent is identified as an agent for use as an inhibitor of bacterial RNA polymerase when there is no change in the activity of the eukaryotic RNA polymerase in the presence of the agent relative to in its absence; and wherein the agent identified inhibits bacterial but not eukaryotic RNA polymerase.

18. A method of identifying an agent that inhibits bacterial growth comprising:
(a) selecting a potential agent by performing rational drug design with the set of atomic coordinates in Table 2, wherein said selecting is performed in conjunction with computer modeling;
(b) contacting the potential agent with a bacterial culture; and
(c) measuring the growth of the bacterial culture under conditions in which the bacterial culture grows in the absence of the agent; wherein a potential agent is identified as an agent that inhibits bacterial growth when there is a decrease in the growth of the bacterial culture in the presence of the agent relative to in its absence.

19. The method of claim 18 further comprising:
(d) preparing a crystal containing a bacterial RNA polymerase formed in the presence of the potential agent, wherein the crystal effectively diffracts X-rays for the determination of the atomic coordinates to a resolution of better than 5.0 Angstroms;
(e) determining the three-dimensional coordinates of the crystal with molecular replacement analysis; and
(f) selecting a second generation agent by performing rational drug design with the three-dimensional coordinates determined for the crystal, wherein said selecting is performed in conjunction with computer modeling.

20. The method of claim 19, further comprising:
(g) contacting the second generation agent with a eukaryotic cell; and
(h) measuring the amount of proliferation of the eukaryotic cell under conditions in which the eukaryotic cell proliferates in the absence of the agent; wherein an agent is identified as an agent for inhibiting bacterial growth when there is no change in the proliferation of the eukaryotic cell in the presence of the agent relative to in its absence; and wherein the agent identified inhibits bacterial growth but not eukaryotic proliferation.

21. A method of obtaining a crystal of an inhibitor bound to a core bacterial RNA polymerase comprising
(a) growing the core bacterial RNA polymerase crystal in a buffered solution containing 40-45% saturated ammonium sulfate, wherein a crystal forms; and
(b) soaking the crystal in 2 M (NH₄)₂SO₄, with the inhibitor; where a crystal of the inhibitor bound to the core bacterial RNA polymerase is formed.

22. The method of claim 21 wherein the inhibitor is rifampicin.

23. The method of claim 21 wherein said growing is performed by a method selected from the group consisting of batch crystallization, vapor diffusion, and microdialysis.

24. A method of identifying a compound that is predicted to inhibit bacterial RNA polymerase comprising:
(a) defining the structure of rifampicin bound to the core RNA polymerase (Rif-RNAP) or a portion of the Rif-RNAP by the atomic coordinates in Table 2; wherein the portion of the Rif-RNAP comprises sufficient structural information to perform step (b); and
(b) identifying a compound that is predicted to inhibit bacterial RNA polymerase; wherein said identifying is performed using the structure defined in step (a).

25. The method of claim 24, further comprising:
(c) contacting the compound with a bacterial RNA polymerase; and
(d) measuring the activity of the bacterial RNA polymerase; wherein the compound is identified as an agent that inhibits bacterial RNA polymerase when there is a decrease in the activity of the bacterial RNA polymerase in the presence of the compound relative to in its absence.

26. The method of claim 25, further comprising:
(e) contacting the compound with a eukaryotic RNA polymerase; and
(f) measuring the activity of the eukaryotic RNA polymerase; wherein the compound is identified as an agent for use as an inhibitor of bacterial RNA polymerase when there is no change in the activity of the eukaryotic RNA polymerase in the presence of the compound relative to in its absence; and wherein the compound identified inhibits bacterial but not eukaryotic RNA polymerase.

27. A method of identifying a compound that is predicted to inhibit bacterial growth comprising:
(a) defining the structure of rifampicin bound to the core RNA polymerase (Rif-RNAP) or a portion of the Rif-RNAP by the atomic coordinates in Table 2; wherein the portion of the Rif-RNAP comprises sufficient structural information to perform step (b); and
(b) identifying a compound that is predicted to inhibit bacterial growth; wherein said identifying is performed using the structure defined in step (a).

28. The method of claim 27, further comprising:
(c) contacting the compound with a bacterial culture; and
(d) measuring the growth of the bacterial culture under conditions in which the bacterial culture grows in the absence of the compound; wherein the compound is identified as an agent that inhibits bacterial growth when there is a decrease in the growth of the bacterial culture in the presence of the compound relative to in its absence.

29. The method of claim 28, further comprising:
(e) contacting the compound with a eukaryotic cell; and
(f) measuring the amount of proliferation of the eukaryotic cell under conditions in which the eukaryotic cell proliferates in the absence of the compound; wherein the compound is identified as an agent for inhibiting bacterial growth when there is no change in the proliferation of the eukaryotic cell in the presence of the compound relative to in its absence; and wherein the compound identified inhibits bacterial growth but not eukaryotic proliferation.

30. A computer having within its memory a representation of rifampicin bound to the core RNA polymerase (Rif-RNAP) or a portion of the Rif-RNAP comprising:
(a) a machine-readable data storage medium comprising a data storage material encoded with machine-readable data, wherein said data comprises structural coordinates from Table 2;
(b) a working memory for storing instructions for processing said machine-readable data;
(c) a central processing unit coupled to said working memory and to said machine-readable data storage medium for processing said machine readable data into a three-dimensional representation of the Rif-RNAP complex or a portion of the Rif-RNAP; and
(d) a display coupled to said central-processing unit for displaying said three-dimensional representation.


SEQUENCE LISTING (30 sequences)

SEQ ID NO: 1 - protein, 1525 aa, Thermus aquaticus
  MOD_RES (1247): Variable amino acid
MKKEVRKVRIALASPEKIRSWSYGEVEKPETINYRTLKPERDGLFDERIFGPIKDYECACGKYKRQRFEGKVCERCGVEVTRSIVRRYRMGHIELATPAAHIWFVKDVPSKIGTLLDLFATELEQVLYFNKYIVLDPKGAVLDGVPVEKRQLLTDEEYRELRYGKQETYPLPAGVDALVKDGEEVVKGQELAPGVVSRMDGVGSLPLPRRVRVDYLRKERAALRIPLSAWVEKEPYRPGEVLAELSEPYLFRAEESGVVELKDLAEGHLIYLRQEEEVVARYFLPAGMTPLVVEGEIVEVGQPLAEGKGLLRLPRHMTAKEVEAEEEGDSVHLTLFLEWTEPKDYKVAPHMNVIVPEGAKVQAGEKIVAAIDPEEEVIAQAEGVVHLHEPASILVVKARVYPFEDDVEVTTGDRVAPGDVLADGGKVKSEIYGRVEVDLVRNVVRVVESYDIDARMGAEAIQELLKELDLEKLERELLEEMKHPSRARRAKARKRLEVVRAFLDSGNRPEWMILEAVPVLPPDLRPMVQVDGGRFATSDLNDLYRRLINRNNRLKKLLAQGAPEIIIRNEKRMLQEAVDAVIDNGRRGSPVTNPGSERPLRSLTDILSGKQGRFRQNLLGKRVDYSGRSVIVVGPQLKLHQCGLPKRMALELFKPFLLKKMEEKAFAPNVKAARRMLERQRDIKDEVWDALEEVIHGKVVLLNRAPTLHRLGIQAFQPVLVEGQSIQLHPLVCEAFNADFDGDQMAVHVPLSSFAQAEARIQMLSAHNLLSPASGEPLAKPSRDIILGLYYITQVRKEKKGAGMAFATPEEALAAYERGEVALNAPIVVAGRETSVGRLKFVFANPDEALLAVAHGLLDLQDVVTVRYLGRRLETNPGRILFARIVGEAVGDEKVAQELIQMDVPQEKNSLKDLVYQAFLRLGMEKTARLLDALKYYGFTLSTTSGIITIGIDDAVIPEEKQRYLEEADRKLRQIEQAYEMGFLTDRERYDQVIQLWTETTEKVTQAVFNNFEENYPFNPLYVMAQSGARGNPQQIRQLCGMRGLMQKPSGETFEVPVRSSFREGLTVLEYFISSHGARKGGADTALRTADSGYLTRKLVDVAHEIVVREADCGTTKYISVPLFQMDEVTRTLRLRKRSDIESGLYGRVLAREVEALGRRLEEGRYLSLEDVHFLIKAAEAGEVREVPVRSPLTCQTRYGVCQKCYGYDLSMARPVSIGEAVGVVAAESIGEPGTQLTMRTFHTGGXAVGTDITQGLPRVIELFEARRPKAKAVISEIDGVVRIEEGEDRLSVFVESEGFSKEYKLPKDARLLVKDGDYVEAGQPLTRGAIDPHQLLEAKGPEAVERYLVDEIQKVYRAQGVKLHDKHIEIVVRQMLKYVEVTDPGDSPLLEGQVLEKWDVEALNERLIAEGKVPVAWKPLLMGVTKSALSTKSWLSAASFQNTTHVLTEAAIAGKKDELIGLKENVILGRLIPAGTGSDFVRFTQVVDQRTLKAIEEARKEAVEAKEKEAPRRPVRREQPGKGL

SEQ ID NO: 2 - protein, 1119 aa, Thermus aquaticus
  MOD_RES (695)..(696): Variable amino acid
MKIKRFGRIREVIPLPPLTEIQVESYKKALQADVPPEKRENVGIQAAFKETFPIEEGDKGKGGLVLDFLEYRIGDPPFSQDECREKDLTYQAPLYARLQLIHKDTGLIKEDEVFLGHLPLMTEDGSFIINGADRVIVSQIHRSPGVYFTPDPARPGRYIASIIPLPKRGPWIDLEVEASGVVTMKVNKRKFPLVLLLRVLGYDQETLVRELSAYGDLVQGLLDEAVLAMRPEEAMVRLFTLLRPGDPPKKDKALAYLFGLLADPKRYDLGEAGRYKAEEKLGVGLSGRTLVRFEDGEFKDEVFLPTLRYLFALTAGVPGHEVDDIDHLGNRRIRTVGELMADQFRVGLARLARGVRERMVMGSPDTLTPAKLVNSRPLEAALREFFSRSQLSQFKDETNPLSSLRHKRRISALGPGGLTRERAGFDVRDVHRTHYGRICPVETPEGANIGLITSLAAYARVDALGFIRTPYRRVKNGVVTEEVVYMTASEEDRYTIAQANTPLEGDRIATDRVVARRRGEPVIVAPEEVEFMDVSPKQVFSLNTNLIPFLEHDDANRALMGSNMQTQAVPLIRAQAPVVMTGLEERVVRDSLAALYAEEDGEVVKVDGTRIAVRYEDGRLVEHPLRRYARSNQGTAFDQRPRVRVGQRVKKGDLLADGPASEEGFLALGQNVLVAIMPFDGYNFEDAIVISEELXXRDFYTSIHIERYEIEARDTKLGPERITRDIPHLSEAALRDLDEEGIVRIGAEVKPGDILVGRTSFKGEQEPSPEERLLRSIFGEKARDVKDTSLRVPPGEGGIVVGRLRLRRGDPGVELKPGVREVVRVFVAQKRKLQVGDKLANRHGNKGVVAKILPVEDMPHLPDGTPVDVILNPLGVPSRMNLGQILETHLGLAGYFLGQRYISPVFDGATEPEIKELLAEAFNLYFGKRQGEGFGVDKREKEVLARAEKLGLVSPGKSPEEQLKELFDLGKVVLYDGRTGEPFEGPIVVGQMFIMKLYHMVEDKMHARSTGPYSLITQQPLGGKAQFGGQRFGEMEVWALEAYGAAHTLQEMLTIKSDDIEGRNAAYQAIIKGEDVPEPSVPESFRVLVKELQALALDVQTLDEKDNPVDIFEGLASKR

SEQ ID NO: 3 - protein, 313 aa, Thermus aquaticus
MLESKLKAPVFTATTQGDHYGEFVLEPLERGFGVTLGNPLRRILLSSIPGTAVTSVYIEDVLHEFSTIPGVKEDVVEIILNLKELVVRFLDPRWRTTLILRAEGPKEVRAVDFTPSADVEIMNPDLHIATLEEGGKLYMEVRVDRGVGYVPAERHGIKDRINAIPVDAIFSPVRRVAFQVEDTRLGQRTDLDKLTLRIWTDGSVTPLEALNQAVAILKEHLNYFANPEASLLPTPEVSKGEKRESAEEDLDLPLEELGLSTRVLHSLKEEGIESVRALLALNLKDLRNIPGIGERSLEEIRQALAKKGFTLKE

SEQ ID NO: 4 - protein, 99 aa, Thermus aquaticus
MAEPGIDKLFGMVDSKYRLTVVVAKRAQQLLRHRFKNTVLEPEERPKMRTLEGLYDDPNAVTWAMKELLTGRLFFGENLVPEDRLQKEMERLYPTEEEA

SEQ ID NO: 5 - protein, 6 aa, Escherichia coli
  MOD_RES (4): Val or Phe
RVIXSQ

SEQ ID NO: 6 - protein, 35 aa, Escherichia coli
  MISC_FEATURE (3)..(7): residues may or may not be present or may be replaced by Val
FFGSSQLXXFMXQDQNNPLXEITXKRXIXXXXPGG

SEQ ID NO: 7 - protein, 14 aa, Escherichia coli
  MOD_RES (2): Pro or Thr
EXXEGPNIGLXXSL

SEQ ID NO: 8 - protein, 7 aa, Escherichia coli
  MOD_RES (4): His or Arg
NMQXQAV

SEQ ID NO: 9 - protein, 6 aa, Thermus aquaticus
RVIVSQ

SEQ ID NO: 10 - protein, 33 aa, Thermus aquaticus
FFSRSQLSQFKDETNPLSSLRHKRRISALGPGG

SEQ ID NO: 11 - protein, 14 aa, Thermus aquaticus
ETPEGANIGLITSL

SEQ ID NO: 12 - protein, 7 aa, Thermus aquaticus
NMQTQAV

SEQ ID NO: 13 - protein, 6 aa, Mycobacterium tuberculosis
  MOD_RES (4): Val or Phe
RVVXSQ

SEQ ID NO: 14 - protein, 34 aa, Mycobacterium tuberculosis
  MOD_RES (3): Asp or Gly
FFXTSXXXXXXXQXNPXXGLXXXRRNLXAXGPGG

SEQ ID NO: 15 - protein, 14 aa, Mycobacterium tuberculosis
ETPEGPNIGLIGSL

SEQ ID NO: 16 - protein, 7 aa, Mycobacterium tuberculosis
NMQRQAV

SEQ ID NO: 17 - protein, 6 aa, Saccharomyces cerevisiae
KVLIAQ

SEQ ID NO: 18 - protein, 31 aa, Saccharomyces cerevisiae
MSSRAGVSQVLNRYTYSSTLSHLRRTNTPIG

SEQ ID NO: 19 - protein, 14 aa, Saccharomyces cerevisiae
ETPEGQACGLVKNL

SEQ ID NO: 20 - protein, 7 aa, Saccharomyces cerevisiae
AMGKQAM

SEQ ID NO: 21 - protein, 6 aa, Homo sapiens
KVLIAQ

SEQ ID NO: 22 - protein, 31 aa, Homo sapiens
HQARAGVSQVLNRLTFASTLSHLRRLNSPIG

SEQ ID NO: 23 - protein, 14 aa, Homo sapiens
ETPEGHAVGLVKNL

SEQ ID NO: 24 - protein, 7 aa, Homo sapiens
AMGKQAM

SEQ ID NO: 25 - protein, 6 aa, Drosophila melanogaster
KVLIAQ

SEQ ID NO: 26 - protein, 31 aa, Drosophila melanogaster
HQARAGVSQVLNRLTFASTLSHLRRVNSPIG

SEQ ID NO: 27 - protein, 14 aa, Drosophila melanogaster
ETPEGAAVGLVKNL

SEQ ID NO: 28 - protein, 7 aa, Drosophila melanogaster
AMGKQAM

SEQ ID NO: 29 - protein, 4 aa, Escherichia coli
YWIV

SEQ ID NO: 30 - DNA, 19 bp, Artificial Sequence
  Description of Artificial Sequence Synthetic polynucleotide sequence
tgaaattgtcggcgattta